(19) United States
(12) Patent Application Publication
(10) Pub. No.: US 2008/0193935 A1
(43) Pub. Date: Aug. 14, 2008

(54) Detection of Dna Sequence Motifs in Ruminants

(75) Inventors: Kylie Munyard, Stoneville (AU); David Groth, Alexander Heights (AU); Keith Gregg, West Perth (AU)

Correspondence Address: Davidson, Davidson & Kappel, LLC, 485 7th Avenue, 14th Floor, New York, NY, 10018, US

(73) Assignee: Murdoch University, Murdoch (AU)

(21) Appl. No.: 11/885,101

(22) PCT Filed: Feb. 24, 2006
(86) PCT No.: PCT/AU06/00240

§ 371 Date: Dec. 28, 2007

(30) Foreign Application Priority Data
Feb. 24, 2005 (AU) 2005900848

(51) Int. Cl. C12Q 1/68

(52) U.S. Cl. 435/6

(57) ABSTRACT

A method for detecting a repeat element in a target ruminant nucleic acid sequence, the method comprising the steps of: (a) contacting a nucleic acid probe capable of hybridizing with a nucleotide sequence flanking said element; and (b) detecting the complex formed between the probe and the target nucleic acid wherein the repeat elements are formed of repeating nucleotide sequences of at least (3) nucleotides.

### FIELD OF THE INVENTION

The present invention relates to the detection of DNA sequence motifs and their use in genotyping ruminant animals. More particularly, the invention relates to the use of tri-, tetra-, penta- and hexa-nucleotide repeating sequences for genotyping ruminant animals.

### BACKGROUND ART

Generally, genotyping of ruminants such as sheep and cattle is performed by analysis of variations that occur in regions of repeating dinucleotide sequences within the genomic DNA or by analysing variations that modify the length of a restriction fragment (RFLPs). Commercially available kits for these types of analysis are available and are currently used for establishing parentage of animals within a population.

However, methods used to identify and to type RFLPs are relatively wasteful of materials, effort, and time. Moreover, RFLP markers are costly and time-consuming to develop and assay in large numbers.

Furthermore, dinucleotide repeat sequences are prone to “stuttering” during in vitro amplification processes such as polymerase chain reaction. This stuttering results in a single original fragment being amplified as two or more fragments of different lengths. The amplification products usually appear on an electrophoretic gel, or capillary electrophoretic analysis as additional bands or peaks, referred to as shadow bands or shadow peaks. The presence of shadow peaks makes the automated analysis of dinucleotide microsatellites imprecise.

In order to accurately determine the copy number of a dinucleotide repeat motif that has shadow peaks, a skilled operator must manually review the sequence data and make a determination of the true repeat number. This has led to genotyping service providers providing either low-cost services with doubtful precision (as the sequences have not been manually reviewed to correct errors due to shadow peaks), or services with relatively high precision but an associated high cost due to the costs involved in manual checking. Several studies have shown error rates of approximately 10% (Visscher et al (2002) J Dairy Science 85: 2368-2375) and even as high as 36% (Baron et al (2002) Genetics and Molecular Biology 25:389-394).

Previous studies in ruminants failed to find the tetranucleotide GATA repeat element in the genomes of sheep or cattle. A few repeat regions have been located in sheep and cattle. However, these repeat regions have not been used for genotyping. Thus, there is a need for an alternative method for genotyping in ruminants that can be automated and which permits relatively accurate high throughput analysis.

### SUMMARY OF THE INVENTION

The present invention provides a method for detecting a repeat element in a target ruminant nucleic acid sequence, the method comprising the steps of:
(a) contacting a nucleic acid probe capable of hybridizing with a nucleotide sequence flanking said element; and
(b) detecting the complex formed between the probe and the target nucleic acid.

wherein the repeat elements are formed of repeating nucleotide sequences of at least 3 nucleotides.

The present invention also provides a method for detecting a plurality of repeat elements in a target ruminant nucleic acid sequence, the method comprising the steps of:
(a) contacting a plurality of nucleic acid probes capable of hybridizing with nucleotide sequences flanking said elements; and
(b) detecting the complexes formed between the probes and the target nucleic acid.

The present invention further provides a method for detecting a repeat element in a target ruminant nucleic acid sequence, the method comprising the steps of:
(a) contacting a nucleic acid probe capable of hybridizing with a nucleotide sequence flanking said element; and
(b) detecting the complex formed between the probe and the target nucleic acid using DNA amplification.

The methods of the present invention can be applied to genotyping. Thus, the present invention also provides a method for characterising a repeat element in a target ruminant nucleic acid sequence, the method comprising the steps of:
(a) contacting a nucleic acid probe capable of hybridizing with a nucleotide sequence flanking said element;
(b) extending the complexes formed between the probe and the target nucleic acid and amplifying the sequence containing the repeat element; and
(c) characterising the repeat element using the amplification products.

The methods herein can be applied to analyse genetic information. Thus, the present invention also provides a method of detecting an association between a genotype and a phenotype in a ruminant using a repeat element in a target ruminant nucleic acid, the method comprising the steps of:
(a) contacting a nucleic acid probe capable of hybridizing with a nucleotide sequence flanking said element;
(b) extending the complexes formed between the probe and the target nucleic acid and amplifying the sequence containing the repeat element;
(c) characterising the repeat element using the amplification products;
(d) determining the frequency of the repeat element In a trait positive population of ruminants;
(e) determining the frequency of the repeat element in a control population of ruminants; and
(f) determining whether a statistically significant association exists between said genotype and said phenotype.

The methods of the present invention may be carried out using kits. Thus, the present invention also provides a kit for detecting a repeat element in a target ruminant nucleic acid sequence, the kit comprising:
(a) a nucleic acid probe capable of hybridizing with a nucleotide sequence flanking said element; and
(b) means for detecting the complex formed between the probe and the target nucleic acid.

The present invention still further provides a method for identifying a repeat element in a ruminant nucleic acid sample, the method comprising the steps of.
(a) contacting a nucleic acid probe or a plurality of nucleic acid probes, designed to hybridise to repeat elements with at least 3 repeats, with the sample; and
(b) detecting the hybrid complex formed between the probe and nucleic acid sample.

### BRIEF DESCRIPTION OF THE DRAWINGS

FIG. 1 shows a gel of 16 sheep samples, amplified using primers BOS3.4RF:5′AAgCAAAATgCCTTACACAT3′ and BOS3.4RR-0.5A GCATCAGCTCAAGAACATT3′ and analysed on a LiCor DNA Fragment analyzer.

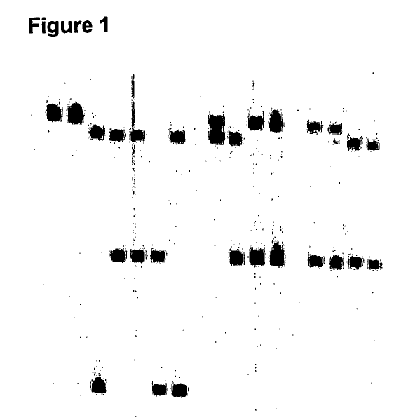

FIG. 2 shows a gel of DNA samples from 9 cattle amplified using primers BOS3.4RF: 5A AGCAAAATGCCTTACACAT3′ and BOS3.4RR: 5A GCATCAGCTCAAGAACATT3′ and analysed on a LiCor DNA Fragment analyzer.

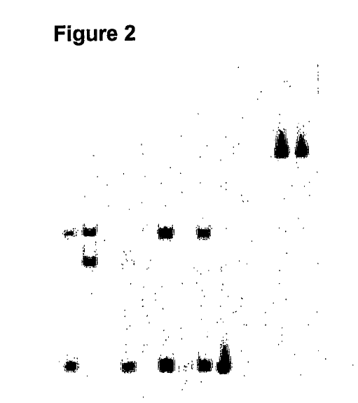

### DETAILED DESCRIPTION OF THE INVENTION

Methods for Detecting a Repeat Element

The present invention provides a method for detecting a repeat element in a target ruminant nucleic acid sequence, the method comprising the steps of:
a) contacting a nucleic acid probe capable of hybridizing with a nucleotide sequence flanking said element; and
b) detecting the complex formed between the probe and the target nucleic acid.

The present invention is based on the surprising discovery that ruminants possess repeat elements of at least 3 nucleotides that may be used for genotyping.

The repeat elements of the present invention are formed of repeating nucleotide sequences of at least 3 nucleotides and more preferably at least 4, 5 or 6 nucleotides. The repeat elements include microsatellites, repeat motifs, simple sequence repeats (SSR), short tandem repeats (STR) and variable number tandem repeat (VNTR).

Preferably, the repeat elements comprise a sequence selected from the group of sequences in Tables 1 to 3 hereunder.

TABLE 1

| Motif | | | | |
|---|---|---|---|---|
| phase 1 | Phase 2 | Phase 3 | Phase 4 | Complement phases 5′-3′ |
| 1. AGC | GCA | CAG | — | GCT, TGC, CTG |
| 2. AGG | GGA | GAG | — | CCT, TCC, CTC |
| 3. AGT | GTA | TAG | — | ACT, TAC, CTA |
| 4. AGA | GAA | AAG | — | TCT, TTC, CTT |
| 5. ACC | CCA | CAC | — | GGT, TGG, GTG |
| 6. ACG | CGA | GAC | — | CGT, TCG, GTC |
| 7. ACA | CAA | AAC | — | TGT, TTG, GTT |
| 8. ATC | TCA | CAT | — | GAT, TGA, ATG |
| 9. ATA | TAA | AAT | — | TAT, TTA, ATT |
| 10. GGC | GCG | CGG | — | CCG, CGC, CCG |
| 11. TAGA | AGAT | GATA | ATAG | TCTA, ATCT, TATC, CTAT |
| 12. CTGT | TGTC | GTCT | TCTG | ACAG, GACA, AGAC, CAGA |
| 13. TTTC | TTCT | TCTT | CTTT | GAAA, AGAA, AAGA, AAAG |
| 14. TAGC | AGCT | GCTA | CTAG | S GCTA, AGCT, TAGC, CTAG |
| 15. TTGC | TGCT | GCTT | CTTG | GCAA, AGCA, AAGC, CAAG |
| 16. GGCA | GCAG | CAGG | AGGC | TGCC, CTGC, CCTG, GCCT |
| 17. GGGC | GGCG | GCGG | CGGG | GCCC, CGCC, CCGC, CCCG |
| 18. GGCC | GCCG | CCGG | CGGC | GGCC, CGGC, CCGG, GCCG |
| 19. | GGAG | GAGG | AGGG | TCCC, CTCC, CCTC, CCCT |
| GGGA | | | | |
| 20. GGGT | GGTG | GTGG | TGGG | ACCC, CACC, CCAC, CCCA |
| 21. ACGT | CGTA | GTAC | TACG | ACGT, TACG, GTAC, CGTA |
| 22. TCGA | CGAT | GATC | ATCG | TCGA, ATCG, GATC, CGAT |
| 23. TGCA | GCAT | TGCA | GCAT | TGCA, ATGC, TGCA, ATGC |
| 24. TACA | ACAT | CATA | ATAC | TGTA, ATGT, TATG, GTAT |
| 25. | GAAG | AAGG | AGGA | TTCC, CTTC, CCTT, TCCT |
| GGAA | | | | |
| 26. GGAC | GACG | ACGG | CGGA | GTCC, CGTC, CCGT, TCCG |
| 27. TCAT | CATT | ATTC | TTCA | ATGA, AATG, GAAT, TGAA |
| 28. TTTG | TTGT | TGTT | GTTT | CAAA, ACAA, AACA, AAAC |
| 29. TTTA | TTAT | TATT | ATTT | TAAA, ATAA, AATA, AAAT |
| 30. AACG | ACGA | CGAA | GAAC | CGTT, TCGT, TTCG, GTTC |
| 31. AACC | ACCA | CCAA | CAAC | GGTT, TGGT, TTGG, GTTG |
| 32. ACTG | CTGA | TGAC | GACT | CAGT, TCAG, GTCA, AGTC |
| 33. AACT | ACTA | CTAA | TAAC | AGTT, TAGT, TTAG, GTTA |
| 34. AGCT | GCTA | CTAG | TAGC | AGCT, TAGC, CTAG, GCTA |
| 35. TTGA | TGAT | GATT | ATTG | TCAA, ATCA, AATC, CAAT |
| 36. GGAT | GATG | ATGG | TGGA | ATCC, CATC, CCAT, TCCA |
| 37. GCGT | CGTG | GTGC | TGCG | ACGC, CACG, GCAC, CGCA |
| 38. CACT | ACTC | CTCA | TCAC | AGTG, GAGT, TGAG, GTGA |
| 39. CAGC | AGCC | GCCA | CCAG | GCTG, GGCT, TGGC, CTGG |
| 40. AAGT | AGTA | GTAA | TAAG | ACTT, TACT, TTAC, CTTA |
| 41. ACAT | CATA | ATAC | TACA | ATGT, TATG, GTAT, TGTA |
| 42. TTAA | TAAT | AATT | ATTA | TTAA, ATTA, AATT, TAAT |

TABLE 2

| Motif phase 1 | | | | |
|---|---|---|---|---|
| Complement phases | | | | |
| (5′-3′) | Phase 2 | Phase 3 | Phase 4 | Phase 5 |
| 43. AAAAC | AAACA | AACAA | ACAAA | CAAAA |
| 44. GTTTT | TGTTT | TTGTT | TTTGT | TTTTG |
| 45. AAAAG | AAAGA | AAGAA | AGAAA | GAAAA |
| 46. CTTTT | TCTTT | TTCTT | TTTCT | TTTTC |
| 47. AAAAT | AAATA | AATAA | ATAAA | TAAAA |
| 48. TTTTA | TTTAT | TTATT | TTTAT | TTTTA |
| 49. AAACC | AACCA | ACCAA | CCAAA | CAAAC |
| 50. GGTTT | TGGTT | TTGGT | TTTGG | GTTTG |
| 51. AAACG | AACGA | ACGAA | CGAAA | GAAAC |
| 52. CGTTT | TCGTT | TTCGT | TTTCG | GTTTC |
| 53. AAAGC | AAGCA | AGCAA | GCAAA | CAAAG |
| 54. GCTTT | TGCTT | TTGCT | TTTGC | CTTTG |
| 55. AAATC | AATCA | ATCAA | TCAAA | CAAAT |
| 56. GATTT | TGATT | TTGAT | TTTGA | ATTTG |
| 57. AAACT | AACTA | ACTAA | CTAAA | TAAAC |
| 58. AGTTT | TAGTT | TTAGT | TTTAG | GTTTA |
| 59. AAAGG | AAGGA | AGGAA | GGAAA | GAAAG |
| 60. CCTTT | TCCTT | TTCCT | TTTCC | CTTTC |
| 61. AAAGT | AAGTA | AGTAA | GTAAA | TAAAG |
| 62. ACTTT | TACTT | TTACT | TTTAC | CTTTA |
| 63. AAATG | AATGA | ATGAA | TGAAA | GAAAT |
| 64. CATTT | TCATT | TTCAT | TTTCA | ATTTC |
| 65. AAATT | AATTA | ATTAA | TTAAA | TAAAT |
| 66. AATTT | TAATT | TTAAT | TTTAA | ATTTA |
| 67. AACAC | ACACA | CACAA | ACAAC | CAACA |
| 68. GTGTT | TGTGT | TTGTG | GTTGT | TGTTG |
| 69. AACAG | ACAGA | CAGAA | AGAAC | GAACA |
| 70. CTGTT | TCTGT | TTCTG | GTTCT | TGTTC |
| 71. AACAT | ACATA | CATAA | ATAAC | TAACA |
| 72. ATGTT | TATGT | TTATG | GTTAT | TGTTA |
| 73. AACCC | ACCCA | CCCAA | CCAAC | CAACC |
| 74. GGGTT | TGGGT | TTGGG | GTTGG | GGTTG |
| 75. AACCG | ACCGA | CCGAA | CGAAC | GAACC |
| 76. CGGTT | TCGGT | TTCGG | GTTCG | GGTTC |
| 77. AACCT | ACCTA | CCTAA | CTAAC | TAACC |
| 78. AGGTT | TAGGT | TTAGG | GTTAG | GGTTA |
| 79. AACGC | ACGCA | CGCAA | GCAAC | CAACG |
| 80. GCGTT | TGCGT | TTGCG | GTTGC | CGTTG |
| 81. AACGG | ACGGA | CGGAA | GGAAC | GAACG |
| 82. CCGTT | TCCGT | TTCCG | GTTCC | CGTTC |
| 83. AACGT | ACGTA | CGTAA | GTAAC | TAACG |
| 84. ACGTT | TACGT | TTACG | GTTAC | CGTTA |
| 85. AACTC | ACTCA | CTCAA | TCAAC | CAACT |
| 86. GAGTT | TGAGT | TTGAG | GTTGA | AGTTG |
| 87. AACTG | ACTGA | CTGAA | TGAAC | GAACT |
| 88. CAGTT | TCAGT | TTCAG | GTTCA | AGTTC |
| 89. AAGCC | AGCCA | GCCAA | CCAAG | CAAGC |
| 90. GGCTT | TGGCT | TTGGC | CTTGG | GCTTG |
| 91. AAGCG | AGCGA | GCGAA | CGAAG | GAAGC |
| 92. CGCTT | TCGCT | TTCGC | CTTCG | GCTTC |
| 93. AAGCT | AGCTA | GCTAA | CTAAG | TAAGC |
| 94. AGCTT | TAGCT | TTAGC | CTTAG | GCTTA |
| 95. AAGGC | AGGCA | GGCAA | GCAAG | CAAGG |
| 96. CCGTT | TGCCT | TTGCC | CTTGC | CCTTG |
| 97. AAGGG | AGGGA | GGGAA | GGAAG | GAAGG |
| 98. CCCTT | TCCCT | TTCCC | CTTCC | CCTTC |
| 99. AAGGT | AGGTA | GGTAA | GTAAG | TAAGG |
| 100. ACCTT | TACCT | TTACC | CTTAC | CCTTA |
| 101. AAGTC | AGTCA | GTCAA | TCAAG | CAAGT |
| 102. GACTT | TGACT | TTGAC | CTTGA | ACTTG |
| 103. AAGTG | AGTGA | GTGAA | TGAAG | GAAGT |
| 104. CACTT | TCACT | TTCAC | CTTCA | ACTTC |
| 105. AAGTT | AGTTA | GTTAA | TTAAG | TAAGT |
| 106. AACTT | TAACT | TTAAC | CTTAA | ACTTA |
| 107. AATAC | ATACA | TACAA | ACAAT | CAATA |
| 108. GTATT | TGTAT | TTGTA | ATTGT | TATTG |
| 109. AATAG | ATAGA | TAGAA | AGAAT | GAATA |
| 110. CTATT | TCTAT | TTCTA | ATTCT | TATTC |
| 111. AATAT | ATATA | TATAA | ATAAT | TAATA |
| 112. ATATT | TATAT | TTATA | ATTAT | TATTA |
| 113. AATCC | ATCCA | TCCAA | CCAAT | CAATC |
| 114. GGATT | TGGAT | TTGGA | ATTGG | GATTG |
| 115. AATCG | ATCGA | TCGAA | CGAAT | GAATC |
| 116. CGATT | TCGAT | TTCGA | ATTCG | GATTC |
| 117. AATCT | ATCTA | TCTAA | CTAAT | TAATC |
| 118. AGATT | TAGAT | TTAGA | ATTAG | GATTA |
| 119. AATGC | ATGCA | TGCAA | GCAAT | CAATG |
| 120. GCATT | TGCAT | TTGCA | ATTGC | CATTG |
| 121. AATGG | ATGGA | TGGAA | GGAAT | GAATG |
| 122. CCATT | TCCAT | TTCCA | ATTCC | CATTC |
| 123. AATGT | ATGTA | TGTAA | GTAAT | TAATG |
| 124. ACATT | TACAT | TTACA | ATTAC | CATTA |
| 125. AATTG | ATTGA | TTGAA | TGAAT | GAATT |
| 126. CAATT | TCAAT | TTCAA | ATTCA | AATTC |
| 127. ACACC | CACCA | ACCAC | CCACA | CACAC |
| 128. GGTGT | GGGTT | GTGGT | TGTGG | GTGTG |
| 129. ACACG | CACGA | ACGAC | CGACA | GACAC |
| 130. CGTGT | TCGTG | GTCGT | TGTCG | GTGTC |
| 131. ACACT | CACTA | ACTAC | CTACA | TACAC |
| 132. AGTGT | TAGTG | GTAGT | TGTAG | GTGTA |
| 133. ACAGC | CAGCA | AGCAC | GCACA | CACAG |
| 134. GCTGT | TGCTG | GTGCT | TGTGC | CTGTG |
| 135. ACAGG | CAGGA | AGGAC | GGACA | GACAG |
| 136. CCTGT | TCCTG | GTCCT | TGTCC | CTGTC |
| 137. ACAGT | CAGTA | AGTAC | GTACA | TACAG |
| 138. ACTGT | TACTG | GTACT | TGTAC | CTGTA |
| 139. ACATC | CATCA | ATCAC | TCACA | CACAT |
| 140. GATGT | TGATG | GTGAT | TGTGA | ATGTG |
| 141. ACATG | CATGA | ATGAC | TGACA | GACAT |
| 142. CATGT | TCATG | GTCAT | TGTCA | ATGTC |
| 143. ACCAG | CCAGA | CAGAC | AGACC | GACCA |
| 144. CTGGT | TCTGG | GTCTG | GGTCT | TGGTC |
| 145. ACCAT | CCATA | CATAC | ATACC | TACCA |
| 146. ATGGT | TATGG | GTATG | GGTAT | TGGTA |
| 147. ACCCC | CCCCA | CCCAC | CCACC | CACCC |
| 148. GGGGT | TGGGG | GTGGG | GGTGG | GGGTG |
| 149. ACCCG | CCCGA | CCGAC | CGACC | GACCC |
| 150. TGGGC | TCGGG | GTCGG | GGTCG | GGGTC |
| 151. ACCCT | CCCTA | CCTAC | CTACC | TACCC |
| 152. AGGGT | TAGGG | GTAGG | GGTAG | GGGTA |
| 153. ACCGC | CCGCA | CGCAC | GCACC | CACCG |
| 154. GCGGT | TGCGG | GTGCG | GGTGC | CGGTG |
| 155. ACCGG | CCGGA | CGGAC | GGACC | GACCG |
| 156. CCGGT | TCCGG | GTCCG | GGTCC | CGGTC |
| 157. ACCTC | CCTCA | CTCAC | TCACC | CACCT |
| 158. GAGGT | TGAGG | GTGAG | GGTGA | AGGTG |
| 159. ACCTG | CCTGA | CTGAC | TGACC | GACCT |
| 160. CAGGT | TCAGG | GTCAG | GGTCA | AGGTC |
| 161. ACGCC | CGCCA | GCCAC | CCACG | CACGC |
| 162. GGCGT | TGGCG | GTGGC | CGTGG | GCGTG |
| 163. ACGCG | CGCGA | GCGAC | CGACG | GACGC |
| 164. CGCGT | TCGCG | GTCGC | CGTCG | GCGTC |
| 165. ACGCT | CGCTA | GCTAC | CTACG | TACGC |
| 166. AGCGT | TAGCG | GTAGC | CGTAG | GCGTA |
| 167. ACGGC | CGGCA | GGCAC | GCACG | CACGG |
| 168. GCCGT | TGCCG | GTGCC | CGTGC | CCGTG |
| 169. ACGGG | CGGGA | GGGAC | GGACG | GACGG |
| 170. CCCGT | TCCCG | GTCCC | CGTCC | CCGTC |
| 171. ACGGT | CGGTA | GGTAC | GTACG | TACGG |
| 172. ACCGT | TACCG | GTACC | CGTAC | CCGTA |
| 173. ACGTG | CGTGA | GTGAC | TGACG | GACGT |
| 174. CACGT | TCACG | GTCAC | CGTCA | ACGTC |
| 175. ACTCC | CTCCA | TCCAC | CCACT | CACTC |
| 176. GGAGT | TGGAG | GTGGA | AGTGG | GAGTG |
| 177. ACTCG | CTCGA | TCGAC | CGACT | GACTC |
| 178. CGAGT | TCGAG | GTCGA | AGTCG | GAGTC |
| 179. ACTCT | CTCTA | TCTAC | CTACT | TACTC |
| 180. AGAGT | TAGAG | GTAGA | AGTAG | GAGTA |
| 181. ACTGC | CTGCA | TGCAC | GCACT | CACTG |
| 182. GCAGT | TGCAG | GTGCA | AGTGC | CAGTG |
| 183. ACTGG | CTGGA | TGGAC | GGACT | GACTG |
| 184. CCAGT | TCCAG | GTCCA | AGTCC | CAGTC |
| 185. AGACG | GACGA | ACGAG | CGAGA | GAGAC |
| 186. CGTCT | TCGTC | CTCGT | TCTCG | GTCTC |
| 187. AGACT | GACTA | ACTAG | CTAGA | TAGAC |
| 188. AGTCT | TAGTC | CTAGT | TCTAG | GTCTA |
| 189. AGCCC | GCCCA | CCCAG | CCAGC | CAGCC |
| 190. GGGCT | TGGGC | CTGGG | GCTGG | GGCTG |
| 191. AGCCG | GCCGA | CCGAG | CGAGC | GAGCC |
| 192. CGGCT | TCGGC | CTCGG | GCTCG | GGCTC |
| 193. AGCGC | GCGCA | CGCAG | GCAGC | CAGCG |
| 194. GCGCT | TGCGC | CTGCG | GCTGC | CGCTG |
| 195. AGCGG | GCGGA | CGGAG | GGAGC | GAGCG |
| 196. CCGCT | TCCGC | CTCCG | GCTCC | CGCTC |
| 197. AGCCT | GCCTA | CCTAG | CTAGC | TAGCC |
| 198. AGGCT | TAGGC | CTAGG | GCTAG | GGCTA |
| 199. AGGCC | GGCCA | GCCAG | CCAGG | CAGGC |
| 200. GGCCT | TGGCC | CTGGC | CCTGG | GCCTG |
| 201. AGGCG | GGCGA | GCGAG | CGAGG | GAGGC |
| 202. CGCCT | TCGCC | CTCGC | CCTCG | GCCTC |
| 203. AGGGC | GGGCA | GGCAG | GCAGG | CAGGG |
| 204. GCCCT | TGCCC | CTGCC | CCTGC | CCCTG |
| 205. AGGGG | GGGGA | GGGAG | GGAGG | GAGGG |
| 206. CCCCT | TCCCC | CTCCC | CCTCC | CCCTC |
| 207. AGTAT | GTATA | TATAG | ATAGT | TAGTA |
| 208. ATACT | TATAC | CTATA | ACTAT | TACTA |
| 209. ATCCC | TCCCA | CCCAT | CCATC | CATCC |
| 210. GGGAT | TGGGA | ATGGG | GATGG | GGATG |
| 211. ATCCG | TCCGA | CCGAT | CGATC | GATCC |
| 212. CGGAT | TCGGA | ATCGG | GATCG | GGATC |
| 213. ATCCT | TCCTA | CCTAT | CTATC | TATCC |
| 214. AGGAT | TAGGA | ATAGG | GATAG | GGATA |
| 215. ATCGC | TCGCA | CGCAT | GCATC | CATCG |
| 216. GCGAT | TGCGA | ATGCG | GATGC | CGATG |
| 217. ATCGT | TCGTA | CGTAT | GTATC | TATCG |
| 218. ACGAT | TACGA | ATACG | GATAC | CGATA |
| 219. ATCTC | TCTCA | CTCAT | TCATC | CATCT |
| 220. GAGAT | TGAGA | ATGAG | GATGA | AGATG |
| 221. ATCTG | TCTGA | CTGAT | TGATC | GATCT |
| 222. CAGAT | TCAGA | ATCAG | GATCA | AGATC |
| 223. ATCTT | TCTTA | CTTAT | TTATC | TATCT |
| 224. AAGAT | TAAGA | ATAAG | GATAA | AGATA |
| 225. ATGCC | TGCCA | GCCAT | CCATG | CATGC |
| 226. GGCAT | TGGCA | ATGGC | CATGG | GCATG |
| 227. ATGCT | TGCTA | GCTAT | CTATG | TATGC |
| 228. AGCAT | TAGCA | ATAGC | CATAG | GCATA |
| 229. CCCCG | CCCGC | CCGCC | CGCCC | GCCCC |
| 230. CGGGG | GCGGG | GGCGG | GGGCG | GGGGC |
| 231. CCCGG | CCGGC | CGGCC | GGCCC | GCCCG |
| 232. CCGGG | GCCGG | GGCCG | GGGCC | CGGGC |
| 233. CGCGG | GCGGC | CGGCG | GGCGC | GCGCG |
| 234. CCGCG | GCCGC | CGCCG | GCGCC | CGCGC |
| 235. CTCCT | TCCTC | CCTCT | CTCTC | TCTCC |
| 236. AGGAG | GAGGA | AGAGG | GAGAG | GGAGA |
| 237. CTGCT | TGCTC | GCTCT | CTCTG | TCTGC |
| 238. AGCAG | GAGCA | AGAGC | CAGAG | GCAGA |
| 239. CTTCT | TTCTC | TCTCT | CTCTT | TCTTC |
| 240. AGAAG | GAGAA | AGAGA | AAGAG | GAAGA |
| 241. CTTGT | TTGTC | TGTCT | GTCTT | TCTTG |
| 242. ACAAG | GACAA | AGACA | AAGAC | CAAGA |

TABLE 3

| 3-base motifs | 4-base motifs | 5-base motifs | 6-base motifs |
|---|---|---|---|
| ATT | CCCT | ACCCC | ACTTTC |
| AGG | TGGC | CAGTT | |
| GGC | CCTT | ACTGA | |
| AGT | GACA | TGAAA | |
| ACG | GAAT | | |
| GTT | AGAA | | |
| GAA | TAAA | | |
| CAG | GTGG | | |
| TGG | GGGC | | |
| | ATTA | | |
| | GATA | | |
| | TGAA | | |
| | ATGG | | |
| | TCTA | | |
| | ATCC | | |

More preferably, the repeat elements comprise a sequence selected from the group of sequences in Tables 4 hereunder.

TABLE 4

| 3-base motifs | 4-base motifs | 5-base motifs | 6-base motifs |
|---|---|---|---|
| ATT | CCCT | ACCCC | ACTTTC |
| AGG | TGGC | CAGTT | |
| GGC | CCTT | ACTGA | |
| AGT | GACA | TGAAA | |
| ACG | GAAT | | |
| GTT | AGAA | | |
| GAA | TAAA | | |
| | GTGG | | |
| | GGGC | | |
| | ATTA | | |
| | GATA | | |
| | TGAA | | |

Preferably, the method for detecting a repeat element in a target ruminant described above is carried out using probes selected from group described in the results section of any one of Examples 1, 2 or 3. Alternatively, the method may be carried out using probes selected from the group consisting of the nucleotide sequences that are identified by bold, italics and underlining in the clones described in the results section of any one of Examples 1 or 2.

The target ruminant nucleic acid sequence may be varied as there are different locations in the genome that contain repeat elements amenable to detection using the method of the present. Preferably, the target ruminant nucleic acid sequence is selected from the group of DNA sequences in the clones described in the results section of any one of Examples 1, 2, 3 or 4 herein that also represent a separate aspect of the present invention.

The target nucleic acid sequence may comprise a single repeat element or a plurality of repeat elements. When there is a plurality of repeat elements they may comprise the same nucleic acid sequence or they may comprise different nucleic acid sequences. For example, the target ruminant nucleic acid sequence may contain a trinucleotide repeat element and a tetranucleotide repeat element.

When there are a plurality of repeat elements it may be desirous to detect more than one repeat element to provide more detailed information on the genome. Thus, the present invention also provides a method for detecting a plurality of repeat elements in a target ruminant nucleic acid sequence, the method comprising the steps of:
a) contacting a plurality of nucleic acid probes capable of hybridizing with nucleotide,sequences flanking said elements; and
b) detecting the complexes formed between the probes and the target nucleic acid.

Whilst the detection of multiple repeat elements could be done separately it is preferable for the detection of different repeat elements to be carried out simultaneously.

The “ruminant” of the present invention is any ruminant or ruminant-like animal. Ruminants include bovines, ovines, caprines, or cervines, while the ruminant-like animal include llamas, camels, alpacas and vicunas. Preferably, the ruminant of the present application is an ovine or a bovine. Most preferably, the ruminant is sheep or cattle.

The nucleic acid probes referred to herein can be used in the method of the present represent but also represent a separate aspect of the invention. The probes are capable of hybridising to regions of the nucleotide sequence flanking the repeat element.

The term probe used herein is used in the traditional technical sense of the term and/or refers to primers for nucleic acid amplification. Thus, it will be appreciated that when used herein the term “probe” also refers to “primer” insofar as the context permits. Furthermore, probes used in the method described herein include variants that hybridize under stringent hybridization conditions to the particular probes described herein.

Preferably, the probes are isolated, purified, and/or recombinant or synthesised as oligonucleotides. Even more preferably, the probes are complimentary to a sequence flanking a repeat element in any one of the clones described in the results section of any one of Examples 1, 2, 3 or 4 herein.

In one form of the invention, the probe is selected from the group consisting of the probes as described in the results section of any one of Examples 1, 2 or 3. In another form of the present invention the probe is selected from the group consisting of the nucleotide sequences that are identified by bold, italics and underlining in the clones described in the results section of any one of Examples 1 or 2 herein.

The formation of stable hybrids depends on the melting temperature (Tm) of the DNA. The Tm depends on the length of the probe, the ionic strength of the solution and the G+C content. The higher the G+C content of the probe, the higher is the melting temperature because G:C pairs are held by three H bonds whereas A.T pairs have only two. The G+C content in the probes of the invention usually ranges between 10% and 75%, preferably between 35% and 60%, and more preferably between 40% and 55%.

A probe according to the invention is between 8 and 1000 nucleotides in length, or is specified to be at least 8, 12, 15, 18, 20, 25, 35, 40, 50, 60, 70, 80, 100, 250, 500 or 1000 nucleotides in length. More particularly, the length of these probes can range from 8, 10, 15, 20, or 30 to 100 nucleotides, preferably from 10 to 50, more preferably from 15 to 30 nucleotides. Shorter probes tend to lack specificity for a target nucleic acid sequence and generally require cooler temperatures to form sufficiently stable hybrid complexes with the template. Longer probes are expensive to produce and can sometimes self-hybridize to form hairpin structures. The appropriate length for primers and probes under a particular set of assay conditions may be empirically determined by one of skill in the art.

Preferred probes of the present invention have a 3′ end that is complimentary to a fragment of the sequence flanking the repeat element. Such a configuration allows the 3′ end of the probe to hybridize to a selected nucleic acid sequence and dramatically increases the efficiency of the probe for amplification or sequencing reactions.

The 3′ end of the probe of the invention may be located within or at least 2, 4, 6, 8, 10, 12, 15, 18, 20, 25, 50, 100, 250, 500 or 1000 nucleotides upstream of the repeat element.

The probes can be prepared by any suitable method, including, for example, cloning and restriction of appropriate sequences and direct chemical synthesis by a method such as the phosphodiester method of Narang et ai. (1979), the phosphodiester method of Brown et al. (1979), the diethylphosphoramidite method of Beaucage et al. (1981) and the solid support method described in EP 0 707592. Probes are generally nucleic acid sequences or uncharged nucleic acid analogs such as, for example peptide nucleic acids (disclosed in WO92/20702) and morpholino analogs (described in U.S. Pat. Nos. 5,185,444; 5,034,506 and 5,142,047).

The probes may be “non-extendable” in that additional dNTPs cannot be added to the probe. Nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group can be cleaved, replaced or modified. U.S. patent application Ser. No. 07/049,061 filed Apr. 19, 1993 describes modifications, which can be used to render a probe non-extendable.

The probes of the present invention may be labelled and thus further comprise a label detectable by spectroscopic, photochemical, biochemical, immunochemical or chemical means. Useful labels include radioactive substances (³²P, ³⁵S, ³H, ¹²⁵I), fluorescent dyes (5-bromodesoxyuridin, fluorescein, acetylaminofluorene, digoxigenin) or biotin. The probes may be labelled at their 3′ and 5′ ends. Examples of non-radioactive labelling of nucleic acid fragments are described in the French patent No. F7810975 or by Urdea et al (1988) or Sanchez-Pescador et al (1988). In addition, the probes may have structural characteristics such that they allow the signal amplification, such structural characteristics being, for example, branched DNA probes as those described by Urdea et al. (1991) or in the European patent EP 0 225 807 (Chiron).

A label can also be used to capture the probe, so as to facilitate the immobilization of either the probe or its extension product. A capture label is attached to the probe and can be a specific binding member that forms a binding pair with the solid phase reagent's specific binding member (e.g. biotin and streptavidin). Therefore depending upon the type of label carried by a probe, it may be employed to capture or to detect the target DNA.

Further, it will be understood that the probes provided herein may themselves serve as the capture label. For example, in the case where a solid phase reagent's binding member is a nucleic acid sequence, it may be selected such that it binds a complementary portion of a probe to thereby immobilize the probe to the solid phase. In cases where a polynucleotide probe itself serves as the binding member those skilled in the art will recognize that the probe will contain a sequence or “tail” that is not complementary to the target. In the case where a polynucleotide probe itself serves as the capture label at least a portion of the probe will be free to hybridize with a nucleic acid on a solid phase. DNA labelling techniques are well known to the skilled technician.

The probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art.

Suitable methods for immobilizing nucleic acids on solid phases include ionic, hydrophobic, covalent interactions and the like. A solid support, as used herein, refers to any material that is insoluble, or can be made insoluble by a subsequent reaction. The solid support can be chosen for its intrinsic ability to attract and immobilize the capture reagent.

Alternatively, the solid phase can retain an additional receptor that has the ability to attract and immobilize the capture reagent The additional receptor can include a charged substance that is opposite charged with respect to the capture reagent itself or to a charged substance conjugated to the capture reagent.

As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatised plastic, magnetic or non-magnetic metal, glass or silicon surface of a test tube, microtiter well, sheet, bead, microparticle. chip, sheep (or other animal) red blood cells, duracytes and other configurations known to those of ordinary skill in the art.

The probes of the invention can be attached to or immobilized on a solid support individually or in groups of at least 2, 5, 8, 10, 12, 15, 20 or 25 distinct probes of the invention to a single solid support. In addition probes other than those of the invention may be attached to the same solid support as one or more polynucleotides of the invention.

The hybrid complex may be detected in a variety of ways. Ultrasensitive detection methods that do not require amplification are encompassed by the present invention as are methods in which the sequences of interest are directly cloned and then sequenced. However, preferably, the complex is detected using DNA amplification. Thus, the present invention also provides a method for detecting a repeat element in a target ruminant nucleic acid sequence, the method comprising the steps of:
a) contacting a nucleic acid probe capable of hybridizing with a nucleotide sequence flanking said element; and
b) detecting the complex formed between the probe and the target nucleic acid using DNA amplification.

Preferably, the repeat elements are formed of repeating nucleotide sequences of at least 3, at least 4, at least 5 or at least 6 nucleotides. In another form, the repeat elements are formed of repeating nucleotide sequences selected from any one of Tables 1, 2, 3 or 4.

The probe used to form the complex may be selected from group described in the results section of any one of Examples 1, 2 or 3. Alternatively, the probe may be selected from the group consisting of the nucleotide sequences that are identified by bold, italics and underlining in the clones described in the results section of any one of Examples 1 or 2.

DNA amplification techniques utilise the hybrid complex as a source of double stranded DNA for extension. It will be appreciated that a single strand is able to function as “template” for PCR, since the first amplification cycle converts it to a double strand. DNA amplification techniques are known to those skilled in the art and may be selected from the group consisting of: ligase chain reaction (LCR) e.g. EP-A-320 308, WO 93/20227 and EP-A-439 182, the polymerase chain reaction (including PCR, RT-PCR) and techniques such as the nucleic acid sequence based amplification (NASBA) described in Guatelli J. C, et al. (1990), Q-beta amplification e.g. European Patent Application No 4544610, strand displacement amplification as described e.g. EP A 684315 and target mediated amplification as described in WO 93/22461. PCR is the preferred amplification technique used in the present invention. A variety of PCR techniques are familiar to those skilled in the art.

Following DNA amplification the amplification products can be visualised by any convenient means apparent to those skilled in the art. For example, the nucleic acids can be applied to PAGE or some other similar technique that separates the nucleic acids, at least on the basis of size. The detection of complexes can also be carried out using detectable labels bound to either the target or the probe. Typically, complexes are separated from unhybridized nucleic acids and the labels bound to the complexes are then detected. Those skilled in the art will recognize that wash steps may be employed to wash away excess target DNA or probe as well as unbound conjugate. Further, standard heterogeneous assay formats are suitable for detecting the complexes using the labels present on the probes.

Genotvping

Variations in the number of repeats within repeat elements can be used to type individuals and thus establish pedigree and/or parentage. Thus, the present invention also provides a method for characterising a repeat element in a target ruminant nucleic acid sequence, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element; and
c) characterising the repeat element using the amplification products.

Preferably the repeat element is characterised according to the number of repeating nucleotide sequences (repeats) of at least 3, at least 4, at least 5 or at least 6 nucleotides, therein. There are various methods that can be used to determine the number of repeats including: sequencing, hybridisation, electrophoretic separation on the basis of length and single strand conformational polymorphism analysis (SSCP).

Preferably, sequencing is automated. For example, dideoxy terminator sequencing reactions using a dye-primer cycle sequencing protocol can be applied. The results from such reactions can be electronically analysed and thus are particularly amendable to high throughput screening protocols.

Hybridization assays including Southern hybridization, Northern hybridization, dot blot hybridization and solid-phase hybridization can be used. When using hybridisation, allele-specific probes can be used in combinations, with each member of the combination showing a perfect match to a target sequence containing one allele. It will be appreciated that hybridization conditions should be sufficiently stringent so that there is a significant difference in hybridization intensity between alleles. These conditions can be determined by one skilled in the art.

Hybridization assays may also be based on multiple probes (arrays) that rely on the differences in hybridization stability of short oligonucleotides to perfectly matched and mismatched sequence variants. Efficient access to polymorphism information is obtained through a basic structure comprising high-density arrays of oligonucleotide probes attached to a solid support (e.g., a micro-chip) at selected positions. Each DNA chip can contain thousands to millions of individual synthetic DNA probes arranged in a grid-like pattern and miniaturized.

Chip technology has already been applied with success in numerous cases. Chips of various formats can be produced on a customized basis by Affymetrix (GeneChip™), Hyseq (HyChip and HyGnostics), and Protogene Laboratories. In general, these methods employ arrays of oligonucleotide probes that are complementary to target nucleic acid sequence segments from an individual wherein the target sequences include a polymorphic marker. The hybridization data from the scanned array may be analysed to identify which alleles of the DNA repeat region are present in the sample. Hybridization and scanning may be carried out as described in PCT application No. WO 92/10092 and WO 95/11995 and U.S. Pat. No. 5,424,186.

Thus, the present invention also provides a method for characterising a repeat element in a target ruminant nucleic acid sequence, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element; and
c) characterising the repeat element using the amplification products by contacting said amplification products with a chip comprising at least one probe selected from the group consisting of the probes described in the results section of any one of Examples 1, 2 or 3.

The present invention further provides a method for characterising a repeat element in a target ruminant nucleic acid sequence, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element; and
c) characterising the repeat element using the amplification products by contacting said amplification products with a chip comprising at least one probe selected from the group consisting of the nucleotide sequences that are identified by bold, italics and underlining in the clones described in the results section of any one of Examples 1 or 2 herein.

The chips that can be used in the present invention also represent an aspect of the invention. Thus, the present invention also provides a chip comprising at least one probe selected from the group consisting of probes described in the results section of any one of Examples 1, 2 or 3 and the complements thereof. The present invention further provides a chip comprising at least one probe selected from the group consisting of the nucleotide sequences that are identified by bold, italics and underlining in the clones described in the results section of any one of Examples 1 or 2 herein and complements thereof.

Multicomponent integrated systems may also be used to characterise the repeat element. These systems miniaturise and compartmentalise processes such as amplification (e.g. PCR) and capillary electrophoresis reactions in a single functional device. An example of such a technique is disclosed in U.S. Pat. No. 5,589,136 which describe the integration of PCR amplification and capillary electrophoresis in chips.

Integrated systems can be envisaged where microfluidic systems are used. These systems comprise a pattern of microchannels designed onto a glass, silicon, quartz or plastic wafer included on a microchip. The movements of the samples are controlled by electric, electro-osmotic or hydrostatic forces applied across different areas of the microchip to create functional microscopic valves and pumps with no moving parts.

For the present invention the microfluidic system may integrate nucleic acid amplification, sequencing, capillary electrophoresis and a detection method such as laser induced fluorescence detection.

The methods for characterising DNA repeat regions described herein can be applied to pedigree analysis, genotyping case-control populations, in association studies, as well as individuals in the context of tracing products from that animal or detection of alleles of DNA repeat regions which are known to be associated with a given trait, in which case both copies of the DNA repeat region present in individual's genome are investigated to determine the number of repeats within a given repeat element so that an individual may be classified as homozygous or heterozygous for a particular allele.

Genetic Analysis

Various methods are available for the genetic analysis of complex traits. The search for disease-susceptibility genes is conducted using two main methods: the linkage approach in which evidence is sought for co-segregation between a locus and a putative trait locus using family studies and the association approach in which evidence is sought for a statistically significant association between an allele and a trait or a trait causing allele.

In general, the methods described herein may be used to demonstrate a statistically significant corre)aï)on between a genotype and a phenotype in ruminants. More specifically, the repeat elements may be used in parametric and non-parametric linkage analysis methods or identical by descent (IBD) and identical by state (IBS) methods to map genes affecting a complex trait.

Preferably, the methods of the present invention are applied to identify genes associated with detectable traits in ruminants using association studies, an approach which does not require the use of affected pedigrees and which permits the identification of genes associated with complex and sporadic traits. One embodiment of the present invention comprises methods to detect an association between a haplotype and a trait.

Thus, the present invention also provides a method of detecting an association between a genotype and a phenotype in a ruminant using a repeat element in a target ruminant nucleic acid, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element;
c) characterising the repeat element using the amplification products;
d) determining the frequency of the repeat element in a trait positive population of ruminants;
e) determining the frequency of the repeat element in a control population of ruminants; and
f) determining whether a statistically significant association exists between said genotype and said phenotype.

Optionally, said ruminant control population may be a trait negative population, or a random population. The method may be applied to a pooled biological sample derived from each of said populations or performed separately on biological samples derived from each individual in said population or a sub sample thereof.

The repeat elements of the present invention can also be used to identify individuals whose genotype increases their likelihood of developing a detectable trait at a subsequent time. These methods are extremely valuable as they can, in certain circumstances, be used to initiate preventive treatments or to allow detection of warning signs such as minor symptoms in an individual carrying a significant haplotype. The methods can also be used to determine which individuals from a population will possess advantageous characteristics such as increased wool production, finer wool, increased milk production etc

Kits

The methods of the present invention can be conveniently carried out using a kit. Thus, the present invention also provides a kit for detecting a repeat element in a target ruminant nucleic acid sequence, the kit comprising:
a) a nucleic acid probe capable of hybridizing with a nucleotide sequence flanking said element; and
b) means for detecting the complex formed between the probe and the target nucleic acid.

The kit may contain a plurality of probes selected from the group consisting of the probes described in the results section of any one of Examples 1, 2 or 3. Alternatively, the kit may contain a plurality of probes selected from the group consisting of the nucleotide sequences that are identified by bold, italics and underlining in the clones described in the results section of any one of Examples 1 or 2 herein. Preferably, the probe is labelled with a detectable molecule. Even more preferably the probe is immobilized on a substrate.

As indicated above a plurality of probes may be used in the methods of the present invention. Thus, the present invention also provides an array comprising a plurality of probes described herein attached in overlapping areas or at random locations on a solid support.

Alternatively the probes of the invention may be attached in an ordered array wherein each probe is attached to a distinct region of the solid support that does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the probes of the invention. One particular embodiment is known as the Genechips™, and has been generally described in U.S. Pat. No. 5,143,854; PCT publications WO 90/15070 and 92/10092.

These arrays may generally be produced using mechanical synthesis methods or light directed synthesis methods that incorporate a combination of photolithographic methods and solid phase oligonucleotide synthesis (Fodor et al., 1991). The immobilization of arrays of probes on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which describe methods for forming oligonucleotide arrays through techniques such as light-directed synthesis techniques.

In designing strategies aimed at providing arrays of nucleotides immobilized on solid supports, further presentation strategies have been developed to order and display the oligonucleotide arrays on the chips in an attempt to maximize hybridization patterns and sequence information. Examples of such presentation strategies are disclosed in PCT Publications WO 94/12305. WO 94/11530, WO 97/29212 and WO 97/31256.

The means for detecting the complex in the kit can be varied and includes the detecting means described herein. Preferably, the kit comprises one or more of the reagents necessary to carry out DNA amplification such as a polymerase enzyme.

Methods For Pe Novo Identification Qf DNA Repeat Regions

As indicated above, the present invention is based on the identification of a number of repeat elements in the genome of ruminants. Thus, the present invention also provides a method for identifying a repeat element in a ruminant nucleic acid sample, the method comprising the steps of:
a) contacting a nucleic acid probe or a plurality of nucleic acid probes, designed to hybridise to repeat elements with at least 3 repeats, with the sample; and
b) detecting the hybrid complex formed between the probe and nucleic acid sample.

The probes used in this method are designed to hybridise to repeat elements with at least 3 repeats and can be designed according to the repeat element of interest. Preferably, the probe is capable of hybridising to 3 to 10 repeats of a repeat element selected from the repeat elements listed in Tables 1 or 2. More preferably, the probe is capable of hybridizing to 3 to 10 repeats of a repeat element selected from the repeat elements listed in Table 3. Most preferably, the probe is capable of hybridizing to 3 to 10 repeats of a repeat element selected from the repeat elements listed in Table 4.

The nucleic acid sample may be obtained from any ruminant source and include biological samples such as body fluids e.g. blood, serum, plasma, cerebrospinal fluid, urine, lymph fluids, and various external secretions of the respiratory, intestinal and genitourinary tracts, tears, saliva, milk, white blood cells, myelomas and the like; biological fluids such as ruminant cell culture supernatants, fixed tissue specimens including tumour and non-tumour tissue and lymph node tissues; bone marrow aspirates and fixed cell specimens.

The preferred source of ruminant genomic DNA used in the present invention is peripheral venous blood. Techniques to prepare genomic DNA from biological samples are well known to the skilled technician.

General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used herein the term “derived” and “derived from” shall be taken to indicate that a specific integer may be obtained from a particular source albeit not necessarily directly from that source.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may he found within the detailed description of the invention and apply throughout. Unless otherwise defined, all other scientific and technical terms used herein have the same meaning as commonly understood to one of ordinary skill in the art to which the invention belongs.

Where this invention describes particular nucleotide sequences such as probes it will be appreciated that the invention extends to variants of the particular sequences described.

A variant of a nucleotide may be a naturally occurring variant such as a naturally occurring allelic variant or it may be a variant that is not known to occur naturally. Such non-naturally occurring variants of the polynucleotide may be made by mutagenesis techniques, including those applied to polynucleotides, cells or organisms. Generally, differences are limited so that the nucleotide sequences of the reference and the variant are closely similar overall and, in many regions, identical.

Variants of nucleotides according to the invention include, without being limited to, nucleotide sequences which are at least 95% ïdentica] to a nucleotide described herein and preferably at least 99% identical, more particularly at least 99.5% identical, and most preferably at least 99.8% identical to a nucleotide described herein.

A hybridizing nucleic acid according to the invention is one that hybridizes to the polynucleotides of the present invention under highly stringent conditions. The following is an example of stringent hybridization conditions:
hybridization is carried out at 65° C. in the presence of 6×SSC buffer, 5× Denhardt's solution, 0.5% SDS and 100 μg/ml of salmon sperm DNA;
followed by four washing steps:
   two 5 min washes, preferably at 65° C. in a 2×SSC and 0.1% SDS buffer;
   one 30 min wash, preferably at 65° C. in a 2×SSC and 0.1% SDS buffer,
   one 10 min wash, preferably at 65° C. in a 0.1×SSC and 0.1% SDS buffer.

These hybridization conditions are suitable for a nucleic acid molecule of about 20 nucleotides in length. The hybridization conditions described above are to be adapted according to the length of the desired nucleic acid following techniques well known to the one skilled in the art. For example, if an oligonucleotide is made of e.g. CCGG, then the washing temperature may be higher for a 20-base molecule. If it is e.g. AATT, then a lower wash temperature may be required to avoid removing fully hybridised molecules.

The present invention will now be described with reference to the following examples. The description of the examples in no way limits the generality of the preceding description.

### EXAMPLES

### Example 1

### Locating Microsatellites in Sheep DNA

Materials/Methods

A modified version of the method of Hamilton, M. B.; Pincus, E. L.; Di Fiore, A. and Fleischer R. C. 1999, Universal Linker and Ligation Procedures for Construction of Genomic DNA Libraries Enriched for Microsatellites. BioTechniques 27:500-507 was used as summarised hereunder.
1. Sheep chromosomal DNA was digested with two restriction endonucleases adapted to form sticky ends compatible with the 3′ overhang of linkers Eco-top and Eco-bottom.

| | |
|---|---|
| Eco-top: | 5′ CTCGTAGACTGCGTACC 3′ |
| Eco-bottom: | 5′ CATCTGACGCATGGTTAA 3′ |

2. The linkers were annealed to form short double-stranded “linkers” and the linkers were ligated to the digested fragments of chromosomal DNA by ligation reactions.
3. Chromosomal fragments were amplified by polymerase chain reaction, using linker oligonucleotides as primers to make amplification independent of chromosomal sequences.
4. The amplified preparation of the chromosomal DNA fragments was heated to separate the strands and a biotinylated selection probe was added to the mixture and allowed to anneal to the chromosomal fragments.
5. The selection probe (annealed to the chromosomal fragments) was removed from the mixture using magnetic metal nanobeads coated with the complementary affinity binding agent, streptavidin.
6. After washing to remove non-specifically bound DNA, the “captured” chromosomal fragments were eluted by heat denaturation and separated from the capture beads.
7. Eluted fragments were re-amplified using priming sites in the linker molecules and the products ligated to a plasmid cloning vector for cloning in E. coli.
8. Clones were screened by hybridisation to identify those containing the appropriate DNA fragments and then sequenced to establish the identity of the repeating sequence motif and to characterise the flanking DNA for potential priming sites for amplification from the genome.

Results

The following repeats were identified in the clones: ATGG, CCTT, ATCC, AGAA, TGGC, ACCCC, CCCT, GATA, GACA, GTGG, ATTA, TCTA, AGAG and AGG

The entire sequences of the clones are set out hereunder. The primer sequences are underlined, bold and in italics.

KM1 (complete, see KM25 for forward primer for CS06) CS06 (tggc)/
CS01 (acccc)
GAΓCCCACGTGCTACAGAGCCACGAAGCCCATAGGCCTCGCCGATGGAATCCGTGCTCTG [symbol] [symbol]
CAAAACCAACCCGGTCAGCCTCCTCCCGGCCCCGGCCGGGGGGCGGGCGCCGGCGGC
TTTGGTGACTCTAGATAACCTCGGGCCGA [symbol] CCCTTCAAGGAAACTCCTGGGGTGACTCCT
GTCCAGGGAATCATCCAAATGGGCCTGTTTCTGAAAAAGGCCCGAGTCACAGCTGTGACA
GATTCTGTGGATCGTGGCTQGCTQGCTGGCTGGCTGGCTGGCTGGCTGGCTGGCTGSCTG
GCTGGATTCCCATGAGAGTCTGAGGATGGAACACATGGACAGAAAAGCATCCBA TCCCT
TΓGG CAAGAATCGGTCTCGCCTTCTGCGCCTGGTGTCTTTCCTACGTCTGGATGATTCC
CTCCCCCACCCCACCCCACCCCACCCCACCCCGCCCCCGCTCCGCTCCCAGCTTGAAGGT
GCTCTCAAGGTCCCGCCGGAACGCTCTCTTCCTCTCTTCGGAGCGCCCTTCTGAAGGGGA
ACgrrrrCrrCCACGrCATCGCCCCGAGACAGCTTCAGCCTGGCCCTCCCCTCCACCCCC
GCCTCCCTCTCTCCCTCCTGCTCCTCTTCCTCCTCCTCTGAACTCTTGAGCTCTCCTCGC
ACCGGCCTCTCACCCCACACGGTGGCAGTGTTGGCCTAGGTATGCTCAGGCGTCTCCTCC
CCGCATCCCAGTGGACTGCCACTGGCTCTCTCTCGACTGCGTCGTCCTGGGACCATGTGT
TTCCTGGCCCTTTCTGCGGGTGGGGGGAGACCCGGACGGGCCNGGCGGGGGTGTGGGGGA
GCCTGCATGCGGGGGGAAGGGTGGGGGCAGAGAGGAGGAGGAGGAGGTGGNCGAGGAGGA
GGAGGAGCAGGAGGACGAGGAGGAGGAACGACACAACTCCCGAGGTGCCAGTGTGTGCCT
GTGGCCCGGGAAACAGACGACGCACCGGGCTGGCTCCGAAAAGGGGATCCCCGTCCTTTG
CGACCCATACCCTGTGTCCTTGCTATGTCAACATGTCACTCGA [symbol] C
KM2 (complete)
GA [symbol] CTTTCCCGCTNNANGGGGNAGCTTNAGGCCAACGTGTTCACTCTCCTCTTTGGGTTT
CCTCAAGAGGCTTTTTAGCCCCTCTTCCCTTGCTGCCATAAGGGTGGTGTCATCTGCATA
TCTGAGGGGA [symbol] CCGTTTCCGGAAAGACGGATACCCCCACGTCGCTTCTTTCTTTCTTGCT
CCCCGTTTCTCTGGCCGAATTCCAAGTGATTCAGCCTCTTTTCCTCCACTCGTTTTCCTA
CGACACGATCCCCCATGTTGTGCAAAAAAGCGGTTACATCATCGACACTTCGAACGCACT
TGCGGCCCCGGGTTCCTCCCGGGGCTACGCCTGTCTGAGCGTCGCTTGGCGATCGCCGAC
TCACTGAACGGAG
KM6 (complete) CS02
GATCGTGTCGCTCCTTTTCTGTTGTCTACGTGTTTCACGGCGAGTGAGTGAGAGAGTCTT
TCGATGGTTTGCTAGGATGTGTGAATGTCGTGAGACCATGGTACTTGTCAGCCGTGGATG
AACAGAACGGCTTCAGCTTTCAGGGTGATCTCAAGTGCACTTTCCCCACCCAGCGGCGCC
TGCTTGGGTTTGTTGTCTTCGGACTTTGTCACGGTCTCTACCCAGGTTGAGTTGTGTCTT
CTCTCGGTGGGGGTTCCGAGTGTGTCTCCTCCTTTTCCTTTCTTGCTCCTGGGCTTGCTT
GTCTGCGTCrGCrrrCCAAflGrCCrGCrrTGTTCTCCGAGCAGCGCTCGCCTTGGTTTCG
CTTTGCCGGCCCCTCCCTCCCTCCCTCCCTCTCTTTCGGGGGAGGGGGGGCCGGGGGAGT
CTGCGATGCCGCTCGCTGGTGCCCCTCTCTCCGCGGACCCCGGGCCGAGCCCCCACCGCC
CGCCGGCGTCTCCQTGGAATGTCCCCCCAGCACCCCGGAATCGCGTGGGGGAGTGAGTCT
CCTTCGTGGCAGCCTCCTGAGGA
KW18 (complete)
G&TCTCGGGAAGCACAGAAAGCCAGAGAGTTGCATGAACCTGACCGTCACGCTTTCAGAA
GCCAAGGGAACCAGAAATGAGGTTCACTCGCGTGTGGGTCTGTCTTTCCACGGGACGAAT
CCTCTCTTTGAGCAGATGAGGGTTCCGGGGGCCCCGTGGAGCAGAGAGGATAGAGAGTTC
CCTCAGGTCCCCTGCTCCTCCCATGCACGCGCACGCTCCCCAACGGTCCTAGGAACAGCC
TGCCCCAGAGGAGCGTGCTGGCCACAACCCACCTCCACGGAGACGGAGACGGCAGTGTCC
GTCCGCGTCAGTCATCCTCGTCCAGAGTCCCCGGGCCGTGGGCCCTCGCCTTCACGCCTG
GCACCGTCCGTTCTGTAGGTGTGTGTCGAACCTGCCCGGAGCCCTGTGGCATCGTCCCG
KM9 (incomplete, centre missing)
GATCATCNTCNCGCTCCNTNGAANGCNGTCCTCNNCAAAAATGACCCANAGCGCTGCCGG
CNCCTGTCCTACTAGTNGCATGATAAATAANACAGTCATAAGTGCGGCGACGATAGTCAT
GCCCCGCGCCCACCGGJLAGGANCTGACTGGGTTGAAGGCTCTCAAGGGCNTCNGTCGANG
CTCTCNCTTATGCGACTCCTGCATTNNGAAGCANCCNNTTAGTAGGTTGANGCNGTTGAG
CACCNNCGCNNCANGGI-ATGGTGCATGCAAGGAGATGGNGCCCANNAGTCNCNCGGNCAC
GGGGCCTGCCACCATACCCNCGNCGAAACAAGCGCTCATGAGCCCGAAGTGGNGAGCCCG
ATCCAAAGAGTGGACAGGACGGTCAGGTGAGTGCCATATGGAAAGGAAAGGAAGNCAACC
CACNAACACCCTCCCNACGGTGGTTGNGTTCANTCCAAGA [symbol] CAGNTCCTTTGACTAGCGT
TGGTACGACGGCNACCACNNGGGGGATGGAGAAACACAACNGTTGGTTTCTTTTGGACGA
NGAGCCCCCCTCTGTGTGTGTGTGTgTGTGTGTGTGTGTgTgTGtGtgTGTgtgTgAGAg
A.......ACGCCAGAGTTTTCCCGANAGAGAGAGAGAGAGAGAGAGAGACAGAGAGAGAGA
GATGGGGATGGGGATGGGAGGAGGGGTGCGTGGGTGGGGCGGATC
KM11 (complete)
GATCATGTAACTCGCCTTGATCGTTGGGAACCGGAGCTGAATGAAGCCATACCAAACGAC
GAGCGTGACACNACGATGCCTGCAGCAATGGCAACAACGTTGCGCAAACTATTAACTGGC
GAACTACTTACTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGATAAATCTGGA
GCCGGTGAGCGTGGGTCTCGCGGTATCATTGCAGCACTGGGGCCAGATGGTAAGCCCTCC
CGTATCGTAGTTATCTACACGACGGGGAGTCAGGCAACTATGGATGAGCGAAGTGGGCAG
GCAGGGGGCCCCCCGAGCAGACACCTTCCTTCCAAAGAAAGGGAGAACAGACAGACACCC
AGAAGCACAAGGGAGACAACAAATCANCGGCAGGGCTGGGCCGGGCTCGGCTGGGGCTGC
TGGGGGTGGGGGCGGGCTCACGGAAGCACCCCGGGGCGTTCATCTGGACATTGATCGTGT
CGCTCCTTTTCTGTTGTCTACGTGTTTCACGGCGAGTGAGTGAGAGAGTCTTTCGATGGT
TTGCTAGGATGTGTGAATGTCGTGAGACCATGGTACTTGTCAGCCGTGGATGAACAGAAC
GGCTTCAGCTTTCAGGGTGATCTTGGACTGAACACAACCACCGTGGGGAGGGTGTTCGTG
GGTTGGCTTCCTTTCCTTTCCCTATGGCACTCACCTGACCGTACCTGTCCACTCTTTGGA
TCCTCTAGAGTCGACCTGCAGGCATGCAAGCTTGAGTATTCTATAGTGTCANCTAAGNAT
CAANCTT
KM 12 (complete) [symbol]
RCTGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGAAGTGGTCC
TGCAACTTTATCCGCCTCCATCCAGTCTATTAATTGTTGCCGGGAAGCTAGAGTAAGTAG
TTCGCCAGTTAATAGTTTGCGCAACGTTGTTGCCATTGCTGCAGGCATCGTGGTGTCACG
CTCGTCGTTTGGTATGGCTTCATTCAGCTCCGGTTCCCAAG
KW115 (complete) CS03
GATCAATGTGTCCTGCAATTCACATTAATTCTCGCAGCTAGCTGCGTTCTTCATCGACGC
ACGAGCCGAGTGATCTTCACGATAAGGGCAGGGAATATGGAGATGGAGCAGCGACCATCA
GCCCAACACATGAAAATCCTTTCCCCAATGTGGCCCTGAAGGTCTATTGAGTCTTCAGAG
AGTGATCCGTTTCTGGAAAGACGGATACCCCCACGTCGCTTCTTTCTTTCTTGCTCCCCG
TTTCVCTGGCCGAATTCCAAGTGATTCAGCCTCTTTTCCTCCACTCGTTTTCCTACGACA
CGA [symbol] CGATTCCCAAAGAGAACGTTCCTTCACTCAAAAAGTCTAGGATTGCTCCCCTTCAA
GACGACTCTCTTTCCTTTTCTACATTCCAACGACATGGATTCATCTATTCCCAGGTGCCT
AAGGATATGGAGGCCTGGCGGCCATCACGGACTCGACCGTGAGAAAAGCCCTGTGCTCGC
GAAGACTCTCCAGAGACTCCCAGACTCTCTGTGCTGTTTACGGTGGAGAGGGAGCCGACG
CTCGTGTGCGTCGTGGCGGGAGGGTGGGTGACCCTGTCACGCGAGCTAGTCTGTCAGCAG
AGAGGTTTGACCCGAGACGCCCTTGTCACACCCAGGGCCGGGCGTGAGCCGTCATGACTG
GNCCGACACGTGAAACACCCTTCACCCACGTCATTCCTGACCAACCCACTAGACTCATCA
TTTCTAGGTAGACGCTGGCTTTGGGGGGAGAGCTTGGGGAACGGGGGGNTTCCTGAGGCT
T
KM25 CS06
TCGGNACTCTCATGGNTAATCCAGCCAGCCAGCCAGNCAGCCAGCCAGCCAGCCAGCCAG
CCAGCNAGCCNCGATCGTGTCGCTCCTTTTCTGTTGTCTACGTGTTNNACGGNGAGTGAG
TGAGAGAGTCTTTCGATGGTTTGCTAGGATGTGTGAATGTCGTGAGACCATGGTACTRGT
CAGCCGTGGATGAACAGAACGGCTTCAGCTTTCAGGGTGATCCTGAACTCCCACGCCAAG
GGAGGCCCTGTGCGTCCCTGTGTGCTGGAGGACACCGTGCTACCCACATCTTGATCTTGG
ACTGAACACAACCACCGTGGGGAGGGTGTTCGTGGGTTGIGCTTCCTTTCCTTTCCCTATG
GNACTCACCTGACCGTCCTGTCCACTCTTTGGATCCTTGATCTCCCCCTCGCCCTCGAGG
CCATCGGTCGGTCCTTTTCTTTCTCCTCCTCCTGCTCCCCGTCCTCCTACTCACCCTAGT
TTCTCTCCCCGCCTCCCCACTCCCCGCCCCTCCACACACACACACACACACACACACACA
CACACACACACACACACACACACACACACGCAAGTCCCGCTCTCTCAAATGGATCTCTCG
CTGACGGCCGACGTTTTCCTTTCGCCTTCTTTCCTTCCTCCCGTCCTGCTTCCTTTCCCT
TTGAGTGNGTGTGTGNGTGTGTGNGTGTGTGTNTGTGAGTGTGTGTGTGTT
KW127 (complete)
ATCCCCTGGAGAAGGAAATGGCAACCCACTCCAGTACTATTGCCTGGAAAATCCCATGGA
CAGAGGAGCCTGGTAGGCTACAGTCTATGGGGTCGCTAAGAGTTGGACATGACTGAGCGA
CTTCACTTCACTTCACTTCACTTCATAAGGTATTGAAAATGCTGAGTGCTCCATTCCTTT
TAAAGGAATTTAAATGTTTTGTTGTCTTTATTCCTAATGACAAGGGACCATGATGGAATT
TAGACCCACTGTCCGCCCACCTATCCATCCATCCAGGCAGCCACCATCCACCTGTCCATG
ATC
KM30 (complete)
GATCCCATTGCAGCCCCAGCTCTCATCTCCTAAGTGGCTGGGGCGTTTTGTTTACTGTTA
CTCAGCCTCTATTTCCTCACACGTACGTGCAGATATAATGAACACATTCCAGTTGTCTGG
CTGTAGTGTTCAGTTCAGTTCAGTCCAGTCGCTCAGTCATGTCCGACTCTTTGCGACCCT
ATGAATCGCAGCATTCCAGGCCTCCCTGCCCATCCATCTCATGTCCATCCAGTCAGTGAT
GCCATCCAGCCATCTCATCCTCTGTCATCTCTTTCTCCTCCTGCCCCCAATCCTTCCCAG
CATCAGGGTCTTTTCCAATGAGTCAACTCTTCACATGAGGTAGCCAAAGTATTGGAGTTT
CAGCTTTAGCATCAGTCCTTCCAATGAACACCCAGGACTGAΓC
KM31 (complete)
GATCTCTGATAGATAAGCAAAGGTTAGACCTGTCCTCAGAACTTTTCTGTATGCTGTGAA
TGGTTCAGTTCAGTTCAGTCGCTCAGTCGTGTCCGACTCTTTGCGACCTCATGAATTGCA
GCATGCCAGGCCTCCCTGTCCATCACCAGCTCCCGGAGTTCACTCAGACTCATGTTCATT
GAGTTGTAGTTGTACCTTTTACTAAAAGTTAATTACTGTCACACACAAAGCGTAGTACCA
CTTAGTAATCATTTATTAAGTGTTGTTGTTCAGTCGCTAAGTTGTGTCCGATTCTTTGTG
ACCCTAAGGACTGCAGCACGCCAAACTTCTTTGTCCTTCACTATCTCTCAGAGTTTGCTC
AAACTCATGTCCATTGAGTTAGTGATGCCATCCATCCATCCCATCCTCTGTCATCCCCTT
TCTCCTCCCGCCTTCAATCTTTCCCAGCATTAGGGTCTCTTCCAATGAATCGGCTAAATC
TATTCAAATATATCTTTCATTTACATGGTACGCTTCATCCGACTTGGAATGATTCAGAAC
CTTTCTAAAAATAAACACTAGGTAAAGAGTAATTTCCTCCCAGATACACATATGGGGAAA
CAGTAAGAATTCACAGGCAACCCTGGGAGTAAACAGAATGGII-TC
KM32 (complete)
GATCCCATGGAATCGCAGCACGCCTGGCCTCCCTGTTCATCACCATCTCCCAGAGTTCAC
TCAGACTCACGTCCATTGAGNCAGTGATGCCATCCAGCCATCTCATCCTCTGTCATCCCC
TTCTCCTCCTGCCCCCAATCCCTCCCAGCATCAGAGTCTTTTCCAATGAGTCGACTCTTC
GCATGAGGTGGCCAAAGNACTGGAGTTTCAGCTTCAGCATCATTCCTTCCAAAGAAATCC
CAGGGCTGATC
KWI33 (complete)
GATCCCTACATTGTATTTCCTAGAATTTTATAAAAGTAGAATCATATAGTCTGAAAAAAA
TCTTTGTATGGATATATACTTTTATTTCTCTTACGAAGGCAACTTTTTTATGTCTTTGTC
CTCTCTCCCTTCCTTCCTTCCTTCCTAACTTCTCTCTCCCTCTCTCTTTACCATGTCGTT
CTACAATTGTTCTGGTACTATTTGTTGAAAAAGCAAATCACACTTTCAATTTTGTCAAAA
ATGTTTGACACTCTT
KM35 (complete)
GATCCCGTGAACTGCAGCAGTCCTAGCTTCCCTGTCCTTCCCTAGCTCCTAGAGTTTGCT
ACAACTCATGTCAGTTGAGTCAGTGATGCCATCCATCCATCTCATCCTCTGTCTCTCCTG
TCTCCTCTTG
KM37 (complete)
GATCCCATTGCAGCCCCAGCTCTCATCTCCTAAGTGGCTGGGGCGTTTTGTTTACTGTTA
CTCAGCCTCTATTTCCTCACACGTACGTGCAGATATAATGAACACATTCCAGTTGTCTGG
CTGTAGTGTTCAGTTCAGTTCAGTCCAGTCGCTCAGTCATGTCCGACTCTTTGCGACCCT
ATGAATCGCAGCATTCCAGGCCTCCCTGCCCATCCATCTCATGTCCATCCAGTCAGTGAT
GCCATCCAGCCATCTCATCCTCTGTCATCTCTTTCTCCTCCTGCCCCCAATCCTTCCCAG
CATCAGGGTCTTTTCCAATGAGTCAACTCTTCACATGAGGTAGCCAAAGTATTGGAGTTT
CAGCTTTAGCATCAGTCCTTCCAATGAACACCCAGGACTGATC
KM49 (incomplete)
ATGGATGGATGGATGGATGGATGGATGGATGGATGGATGGATGGATGGATGGATGGATGG
ATGGATGGATGGATGGATGGATGGATGGATGGATGGATGGATGGATGGATGGATGGATGG
ATGGATGGATGGATGNNNTNCAGCTAGGNANGCCTTCCTTCCTTCCTTCCTTCCTTCCTT
CCTTCCTTCCTTCNTACTTNNNTTNNTT
KM61 /62/63/64/65 (complete)
GATCCCAGGGACAGACCTAAAACACTGCTTTACACACAGCCTTGGCTTTCACTGTTCAGC
CATCTCTCTCTACCAATGGACAGTGAGTTGTGGGGGTGAGGACCATGCCCATATCATTTC
TACATTTCCACCTCCCAGCAAGGCACCCAGGAGGACCCTGGAATAATCTGTCAGATGGAT
GGAAGGATAGATGGATGGATGGATGGACGGATGGATGGACGGATGGACGGACAGATGAAT
GGATGGATGGACAGATGGATGGGTGGACGGACGGATGGATGATGGATGGACAGATGGATG
GATGGATGGATGGATGGATGGACAGATAGGTGGACAGATGAATGGATGGACAGACAGATG
GATGGATGGACAGACAATGGATAGATGGATGGATGGATGGATGGATGGATGGACAGATGG

[symbol]

KM75 (complete)
GAΓCAATTATTAGAACTCTATTGCATATGTCCAAAAAATTTAAGTAGAGCCATCAGTCCA
GTTCAGTTTAGTTCAGTTCAGTCGCTCAGTCGTGTCTGACTCTTTGCGACCCCATGAATC
GCAGCACGCCAGGCCTCCCTGTCCATCACCAACTCCCGGAGTTCACTCAGACTCACGTTC
ATCAAGTCAGTGATGCCATCCAGCCATCTCATCCTCTGTCGTCCCCTTCTCCTCCTGCCC
TCAATCCCTCCCAGCATCAGGGTCTTTTCCAATGAGTCAACCCTTCTTATGAGGTGCCCA
AAGTACTGGAGTTTCAGCTTTAACATCATTCCTTCCAAAGAAATCCCAGGGCTGAΓCCAA
CCAGTCCATTCTAAAGGAGATCTGTTAGTGCAGGGAGCCCACTGTGTTGCCTGTATGTTC
TGTGTCTTGGTTCAGCCGCTGTGGACCCTGAGTGAGCTCTTCTTTTGGGACGCAGCTACA
GTTGGATTATCTGGGCCACATGCGCTCATCAAGCTTCCCAGTTGGCTCAGTGGTAAAGAA
TCCCCTGCAATGCAGGAGACACAGAAGCCTCGGGTTCAATTCCTGGGTCAGAAAGATC
MNS242 (incomplete)
GATCATATTCAGAAGAAATTATTAAAACCATAAATTTCTATAAGGGAAGCATGGGTTTCC
CTTGTGGCTCAGCTGGTGAAAGAATCCGCCTGCAATGCAGGAGACCTGGGTTCGATCCCT
GGGTTGGGAAGATCCCCTGAAGAAGGAAACGACAGCCCACTCCATTACTAGTGCCTGGAA
AATCCCATGGACGGAAGAGCCTGGTTAGGCTGCAGTCCATGGGATOSTAAAGAGCCAGAC
ACGACTGCGTGACTTCACTTTCACTTTCATAAGGGGAGCATATTAGTTCTAAAGCATTAG
TTAACAACACCTTGCTGATCTTTTTGCAAAATTTCAGAAAATAATTGTATGTGCGCTCTC
TCTCTCTCTCTCTCTCTCTCTCTCTCTCACACACACACACACACACACACACACAGTTTC
TTTTCTGAGGGACCTTGAGAGTAAGTGAΓCTTAATGCTTCCCTTTGCAGACAGCACAATT
CGGGGTGAGGGGGTGTTGTCCATGGTGCTGAAGTTGTCAGGGGCAGAACTAGAAATAATT
TCTTGACTGCAGTCCATTTCTTTTCCGTGTGATTATGTTGCCTCATCCAGTATATTGTGG
GTCAGGGTCAATCTGTTGTCTCCTTTGCTCTGAAATCTCTGAAATGCTCCTAGGGTGCAT
CCTCACGCCAACCAGCAGCTGCTTTCTAAAAGGAGCATTTGAATGCAACTCTGAATCCTG
AGGAGGAAATGGTTTTCACTGTGGTTTGAAATCTTTTCTATACTCTCTCCACCCACGTAT
A
KM85 (incomplete)
ATGGATGGATGGATGGATGGATGGATGGATGGATGGATGGATGGATGGATGGATGGATGG
ATGGATGGATGGATGGATGGATGGATGGATGGATGGATGGATGGATGGATGGATGGATGG
ATGGATGGATGGATGGNNIrøCTGCTAMrølSrNNNCTTGCTTCCTTCCTTCCTTCCTINrNNTN
ISMTNANTfANTiSfNNTNIrøTNNNTNCNTNlSøSIT
KM86 (complete)
AGGCCTTCCTTCCTTCCTTCCTTCCTTA
KM87 (complete)
AAGGAAGGAAGGAAggaaggaAGGGGGAGGTGGAGGGAGGGGTCTCTCTGGCTGTCTCTC
TAGGAGTCTATTCAAGTCAAAGTATGATAGAGCTGGi\hat{ }GGGAACTTGATTCCAATGTGGT
CTAAGCCTGTGCTTTCATGTAg/cATATGAATGGATCTTCTATAGTTGAGGTJyiGGCTCA
a/gAGATGCTTCTCAAAAGTCACACAGCAAGAGTGTTGATATGTCTTCTTGiYTTCTGGg/
tGGAGTGTTCCCTTCCCTACGTTAGGTTTCATTTGAGACATTTCACATTTCCTTCCATAT
GTCCATCCATCCACCCATCCACCCATcATTGCATCTATGGTTCTATCCATCCATCCGccC
aTcCATCGCCATCCACCCATACACCGATCCATCCATCATCCATCTATCCATCATCCATCC
ATCCATCATCCACCCATCCACCCATCATTGCATCTATGGTTCTATCCATCCATCCATCCA
TCCATCCATTGCCATCCACCCATACACCCATCATCCATCCATCCaCCCATTCATCCATCC
aTcCATCCATTcaTTCATTCaTCTATCCATCCaTCCATCCATCCATTCATCACCATCCAc
CCaTCCATCCaTCCaTCCATCCaTA
KM89 (complete)
AAGGAAGGAAGGAAGGA [symbol] KGGAAGGGGGAGGTGGAGGGAGGGGTCTCTCTGGCTGTCTCTC
TAGGAGTCTATTCAAGTCAAAGTATGATAGAGCTGGAAGGGAACTTGATTCCAATGTGGT
CTAAGCCTGTGCTTTCATGTAGATATGAATGGA [symbol] CTTCTATAGTTGAGGTAAGGCTCAGA
GATGCTTCTCAAAAGTCACACAGCAAGAGTGTTGATATGTCTTCTTGATTCTGGTGGAGT
GTTCCCTTCCCTACGTTAGGTTTCATTTGAGACATTTCACATTTCCTTCCATATGTCCAT
CCATCCACCCATCCACCCATCATTGCATCTATGGTTCTATCCATCCATCCACCCATCCAT
CGCCATCCACCCATACACCCATCCATCCATCATCCATCTATCCATCATCCATA
KM92 (complete)
24,GGATGGATGAGTGGATGGAAGGA\hat{ }GGAAAGATGGATGGGTGGGTAAAAGGATGGATGGA
TGGGTGGACAGACGGAAGAAGACAAGAATGGATGAATGCATTCATGCATGCAAGGGTGTG
AGACCGTCATGGGCGCTGGTCAGGGAAGGCTTCJKGGGACTGGACTTGGACTGAACTTGGT
TGAGAGAGAGCCCAGAGTGGTGGGAGTCTCAGGTGTGCTGCGGAGGA [symbol] CCATGACTTTGT
CCACAAGACCATGCTCCCCCCATCCAGCATGTGGTCTTCCAGAGTCACTGACTCAGCTTC
TCTCCTGCTCT&GGACGGAACCC&GGTGCCA&GGAGCTGACCγIGGGG
KW193 (complete)
ATCGATAGATAGATAGATAGACAGATAGAAAATAGACGTATAGATAGATAGATAGATAGA
TAGATAGATAGATAAATAGATAGATAGATAGATAGATAGATAGATAGATAGACAGAGAGA
CAGATAGATACAAAGACAGATAGACAGATAGATAGGTAGACAGACAGACAGATAGGCAGA
TAGATAGATAGATAGACAGATAGGCAGATAGATAGATAGATAGACAGATAGATAGAGAGA
GAGAGAGACAGACAGACAGAGAGACTGACACTAGCTGATGGCGCAATGAAAAGTGATCC
KM94 (complete)
GATAGTTAGATAGACTGGGTGGATGGATGGATGTATGGACAGACAGATAGACTGGATGGA
TGGATGGATGGATGGATGGATGGATAAATAGATAGACTGGGTGGATGGATGGATGGATAG
ATAGACTTGATGGATGGATGGATGGACAGACAGATAAACTAGATGGATGGATGGATGAAT
GGATAGATGGGTAGATAGACTGGGTGGATGGATGGATAGACAGATAGATAGACTGGGTGG
ATGGATGGATGGATGGACAGACAGACTGGATGGATGGATGGATGGATAGATGGGTAGATA
GACTGGGTGGATGGATGGATGGATGGATAGTTAGATAGACTGGGTGGATGGATGTATGGA
TGGACAGACAGATAGACTGGATGGATGGATGGATGGATGGACAGACAGACTGGATGGATG
GATGGATGGATGGATGGATGGGTGGATGGGTAGATAGACTGGGTGGATGGATGGATGGAT
GGATGGATGGATGGATGGATG
KM95 (incomplete)
AGATAGCCJ\hat{ }CCAGCTAGCC&GACAGACAGAAAGACAGCCAGGCAGCCAGACAGACAGAC
AGACAGACAGACAGCCAGGCAGCCTGACAGACAGACAGACAGACAGCCAACCAGCCACAC
AGCGAGGGAACCAGCCAGCTAGACAGCCAACCAGCTAGCCAGACAGACAGAAAGACAgCC
agAcagACAGAcagacaGacaGAcagACagacagaCagCCAACcagaCagaCaGCCagcc
agccagac
KM96 (complete)
atGGATGGATGGATGGACGGGCGGATGGATGGGTGGACGGATGGGCAGATGGATGGATGA
CAGATGGATGGATGGATGGATGGATGGATGGATGGTTGGACAGACAGATGGATAGGCAGA
TAGATGGTTGAATGGACAGATGGATGGATGCATGGATAGATGAATGGATGGATGGACGGA
TGGACAGATGGATGGACGGATAGACGGATGGATGGACAGATGGATGGACAGGTGGACAGA
TGGATGGATGGTGGGTGGATGGATGGATGGATGGATGGACAGATGGATGGACAGAtggat
GGATGGACAGACGGATGGATGGGTGGATGGGCAGATGGATGGATGGATGGATGGGCAGGC
AGGCACTTGGGAACCCACAGGTTTCCCCGGAAGCTACAGGCAGGAGGTGGCATGTATGTG
AATGGTAGATGGGATCTGGGTGAGAGAAAGGACAGAAGGTCACACCTCTGGAGACCCAGT
GAACCGAGGTGCCTGATGGGTTTCTAAG
KM98 (complete)
GATTCAGACAGGCAGAGAGATTATATGTACCAgAAGAAATAgACaGACAGAGAACATATG
TATATaCAGAGACAAACAGGCAGAGATTGTTGTAGAAGAACAGACAGGCAGACAGACAGA
CGGCAAACGAGATTGTGAGGGAGGGACAAAGAACCACAGAGGGATTATAGGCCTGAGGCG
ATGAAGAGTGTGTGTTTGGTGTGAGGTCCTCGAGCGTTGAGTTCCCCAGCAGCACTCGAC
CACTGACCATCTGCCACGCCCCAACCTACTACCCTCCTCCTCCCTCTT
KM 101 (complete)
AAGGGGTCGCTCCTCTTTGCAGCTGCCGTTCATATGTTTGGGGGAGTTTGGCTCTAGAGA
AGCCAGGGTCACGAGTTTAGGCTCCATGATGTGGGGGAGCAGACCAAGAAAGTAATTTGG
TGCTGGTCTACAGCGCCTGGGCAGAGCTCTGTCCATGCCTGCCTTGGTCCTCAGGTGGGA
ATCAGGATGGTTCACTGTAGCTCCCCATGGGTGCAGATAAAACTGCTTAGAGCACCAGCG
TAGAGAGATAGGCAGAAATGATAGAATAGATTAGATATAGAGGATGGGTGGATGGGTTAG
GTGGGTAGTTGCATGCATGGGTTGaGGGGTGGCTTGGTGGATGGATATGAATGGATGGAT
GGTAGCTACGTGGATGGATGTATAGATGGGTGGATAGGTGAATGTAGATGGGTAGATAAT
AGATGGATGGATGGATGATGGATGGATGAATGGG
KM 102 (complete)
GATTCAGACAGACAGAGAGATTATATGTACCAgAAGAAATAGACAGACAGAGAACATATG
TATATACAGAGACAAACAGACAGAGATTGTTGTAGAAGAACAGACAGACAGACAGACAGA
CGGCAAACGAGATTGTGAGGGAGGGACAAAGAACCGCAGAGGGATTATAGGCCTGAGGCG
ATGAAGAGTGTGTGTTTGGTGTGAGGTCCTCGAGCGTTGAGTTCCCCAGCAGCACTCGAC
CACTGACCATCTGCCACGCCCCAACCTACTACCCTCCTCCTCCCTCTT
KM1 04 (complete)
ACACACAGGATAATCTTCGTAATGTCTTCGTAGTATGAGTTGCTTTGTGCGAGCGGTGGT
TACAGAACTGTTTGCCTGTGCAAGACTGGTAGTGGAAGGCTGGAGTGAAAATTCCGAAGT
GGTGCGTCTAATTCTATATTAGCTTCTGTTTTTTCATTATGGGGTCTCTCGTGATGTGGA
AGATAGTGAAACTAAACTACGTTTCAGGATTGTATGGAAGACACGTCTCTCTCTCTCTCT
CTCTCTCTCTCTCTCTCTCAATCTATCTTATCTATCTATCTATCTCACTCTGTCTGTCTA
TCTATCTATCTATCTATCTGTCTATCTgtcTATCT&TCTATCTATCTATCTATCTATCTA
TCTATCTATCTATCTATCTATCTTTCTACTGACTTTCGGC
KM 105 (incomplete)
GATAGTTAGATAGACTGGGTGGATGGATGGATGTATGGACAGACAGATAGACTGGATGGA
TGGATGGATGGATGGATGGATGGATAAATAGATAG&CTGGGTGGATGGATGGATGGATAG
ATAGACTTGATGGATGGATGGATGGACAGACAGGTAAACTAGATGGATGGATGGATGAAT
GGATAGATGGGTAGATAGACAGGGTGGATGGATGGATAGACAGATAGATAGACTGGGTGG
ATGGATGGATGGATGGACAGACAGACTGGATGGATGGATGGATGGATAGATGGGTA_{G}ATA
GACTGGGTGGATGGATGGATGGATGGATAGTTAGATAGACTGGGTGGATGGATGGATGGA
TGGACAGACAGATAGACTGGATGGATGAATGGATGGATGGACAGACAGACTGGATGGATG
GATGGATGGATGGATGGATGGGTAGATAGACTGGGTGGATGGATGGATGGATGGATGGAT
GGATGGATGA
KM1 06 (complete)
CCAATGGATGAATGAGTGGATGGGAGGATAGACAGGgagATGATGCaCTGATAgACGCa/
gTAAAAAGATGGGTGAGTAAATGGATGGATGGGCAGATGGAAGAaTGGatGGatGGGTGG
ATAGAAATATGGGCAGGTAAAGGGAGGAAGGGATGGGGAGACGGATGAATGGATAGGTGG
ATAGGAAGATTGCTGAGTGGATGGATGGATGGGTGGATGGATGAATGGATGATGGACGGT
CCAGTAGCAAGGTGGATGGGCGGGTGGCTAGATGTATGGATGGAGAGGAGTGAATGTcaa
aaGGAAGACC
KM 107 (complete)
ggggatgGAGGAGTGGAACAGTGAATGGACAGCAGCCGAgAGAGAGGAGCAGCTGGAGAT
GGCGGacGatggatgGgCGGGTGGATGGATGGGTGGATGGATGGatGGGcGGATGGaTGA
ATGGGCGGATGGATTAATGGAtGGAtGGAtGGATTAATGGGTGGaTGGATGGATTAATGG
GTGGaTGGGTGGATGAATGGGTGGATGGATTAATGGATGGATGGGTGGGTTAATGGGTGG
ATGGATAAATTAATGGGTGGATGGATGGATTAATGGATGGATGGGTGGATTAatgggtgg
aTGGATGGATGAATGGGCGGATGGatgaatgggCGGATGGATGAATGGGCGGATGGATTA
GTGGGTGGATGGATAGACAGtgaGtGaaTGAgTGAAAGGATGG
KW1108 (complete)
ACCGTTCCCAGTTAAGTAATTCAGCTGTATCGTGACTTGCAGAAGGTAGAGAGAGAGAGA
AAGAGAGAGAGAGAGAGAGAAAGGGAGAAAAGATAGATAGATAGATaGaTAGATAGAGAT
AGAGkGAGGGAGAAAAGGTAGATAGAGAGAGAGAGAGAGAGAGAGAGAGAGAGAGAGAAA
GGGAGAAAAGGTAGATAGATAGATAGATAGATAGAGAGAGGGAGAAAAGGTAGATAGAGA
GAGAGAGAGAGAGAGAGAGAGAGAGGAGAATACATGGCGGAAGTTGAGGCGAAGAGAGga
cagcaGCGAGTGTTTATGTTTGTGCC
KM 109 (complete)
ACTCTCTTAGTTTCTGCGATGAACTCACTATTCTTATCTTTTCAACCGACATGGCTTAGA
CTGGGGCATACCTTCGCCTGTGCCATGGAGGTTACAGTGGAGTAgAAGACAGAGAS-ACAG
ACAGAGAAACAGGCAGACAGACATACAGACAAACAGAGAAACAGATACAAGACAGACAGA
CAGACAGAGCGACAGACGAACAGIkAAAGCAGACAGACAGACAGAGAaACAAACAGATAGA
CAGACTGACAAGCAGAAGC
KM1 10 (complete)
ATCAAACCAGAATATTAATGACGAGTTCTGAATTTTTGGTCTGTCGACCTCTTTTCCTTC
TTTTTTACCTATTTCTTTCCTCAGTGAAGCGAATATAATGTCTATCTGTTTATCTGCCTA
TCTGTCTATCTATCTATCTATCTATCCGTCTGTCTGTCTGTCTACCACGCCTACCATACA
TAAGGTCCCGTGTTCGAGCCCTGGCTGTTGGAGGGCTTGTGTTCTAAAAAAGCGTGCTTT
TATATGCACTGTATTCGTGTGTGTATC
KW1111 (incomplete?)
atGaAAGCACAGGcTTAGACCACATTGGAATCAAGTTCCCTTCCAGCTCTATCATACTTT
GACTtgaatAAACtCCTAGAGAGACAGCCAGAGAGACCCCTCCCTCCACCTCCCCCCTCC
TTCCTTCCTTCCTTCCTTCCTTAATCGAATTCCCGCGGCCGCCATGGcGGCCGfGGAGCAT
GCGACGTCGGGCCCAAtTCGCCCTATAGTGAGTCGTATTACAaTTCACTGGCCGTCGTTT
TACAACGTCgTGACTGGGAAAACCCTGGCGTTACCCAACTTAATCGCCTTGcagcacatc
cCCCTTTCGCCAGCTGGCGtaaT
KM113 (complete)
aGAGAGAGAGACAGACAGAGAGAGAGAGAGAGAGACAGACAGACAGACAGACAGACGGAC
agacagAcAgACAGACAGACAGACAGACAGACAGACAGAcaGacagAGACAGAGACAGTC
AGACAGAGACTGACAGACAGAGACAGAGACAGTCAGACAGAGACAGAGACAGTCAGACAG
AGACTGACAGACAGACAGACAGACAGACAGACAGACAGACAGAGAGTGAGTC
KM1 14 (complete)
ACATATGGATAGTAACTTATATGATGACCAAATGAAGAACAAGAAATATTACGAAGTGAA
AAGAATAATAAAGCAGGCGAACCAAGAGGCTGAGCAGCGTTCATAAAGTCATGATAATCA
TAGACTGACTAATTATGGGATATGAGGGTATTGATGCCTTAAACAGAGAGAGAGAGAGAG
AGAGAGAGAGAGAGAGAGAGACAGAGAGAGAGAGAGAGAGAGACACAGACAGACAGACAG
ACAGACAGACAGACACACAGACAGACAGACAGACAGAGACAGAGACAGAAAGATTTATAA
TGAATGCAATGCACAATAGAGAGGGAGATACTAATAAGTCAGAGAAS-ACACGTAGCATCC
TGAGGCAGACCTACAGATGGAGCAAGTCGGTGTTGTGAATATAAGGAGAGCCC
KW1 115 (complete)
GAGATGAATAGGTGGATGGATGGAGAGATGAATGAATAGATGGATGGATGGATGGATGGA
TGGATGACGGATGGTGATGGGTGGATGATGGGTGGATGACGGGTGGGTGATGGGTGGATA
GATGAATAGGTGGGTGGATGGAGAGATGAATAGGTGGATGGATGGATAGATGGATGAATG
ACTAGATGGGTGATGGATGGATGAATAGATGGATGGATGGAGAGATGAATGAATAGGTGG
ATGGATGGATGAGGGATGGATAGGTGAATAGGTCGATGGATGGACAGATAGATGGATGGA
TGGATGATGGGTGGATGATGGATGAaTagatGGaTGGATGGATGATGGATGGATGAATAG
ATGG&TGGATAGAGAGATGAATGAATAGGcAGATGGATGGATGATGGATGTATAGATGGA
TGGATGAATGAATAGATGGATGGATGGATAAATGGATGGATGCC
KM1 16 (complete)
ATGATGAAGCCGACGCTGAAGGTGAt/ggATGGAGACGCAGATGAATACa/ga/gGGGGA
GAGAGAGAGAGAGAGAGAGAGAGAGAGAGAGAGACAGACAGACAGACAGACAGACAGACA
GACAGACAGACAGACAGAgACAGAGAGACAGAGACAGAGAGAGACAGACAGAGAGACAGA
TGAlr/GACCCCTTGGAAGNNAGACCTTcTCTCAGTGATACNNTCTCNCTaANNGNGACNA
CTCNCTCTCGATGTCACTTCCTACNGACGGAATCTGCTTCTAAACGNANCCNACNTTNAN
NTAAAACTCTCTCTCTACAAACtMNNN
KIW1 18 (complete)
GGATGATGGGATGGATGAGTGGATGATGGGATGAATGGGTGGGTAGATGATAGAATGAAT
GGGTGGGTGGATGATGGGATGGATGGATGGGTGGATGATGGATGGATGGGTGGATGATGG
GATGAATGGATGGGTGGATGATGGGATGGATGGATGGGTGGATGATGAGATGGATGGATG
GGTGGATGATGGGATGGATGGTTGGGTGGGTGATGGGATGGATGGGTGGATGATGGGATG
AATGGGTGGATGATGGCTGGATGACAGGTTGACGATGCTGGATGGGTGGGTAGGAAGGCT
GCTATGCCCTGAGTGTTTGTGCCCCaccGGGTCTCACGTCTGGACTCTGGGACCACCGTC
ACACTCACCTGGGTGTAGGTCTAtCtGGAAATTAGCGTCGTGAGGGTTTCTGGCTTCTGT
CCTGCGAGGTGACTGACCCAGTAGTCTAGTTTGTCCCCAGGAGCTTCTGTGCACTGAGGC
ATCCTCGCCGCCCCAGTAACTAAGCAGCACCCCACTGTCAGGTAAGGGG
KM19 (complete)
GATCaTAgCATCAGTGGCAAATGAgATTCTTAAGAAATTGCTGTCTGt/gCTCAGTCTGt
CTGTCTGTCTGTCTGTCTCTCTGTCTGTCTGTCTCTGTCTGTCTGTCTGtCTGTCTGTCT
GTCTGTCTGTCTGTCTATCTGTCTGTCTGTCTGTCTGTCTGTCTGTCTGTCTGTCTGTCT
CTCTCTCTCTCTCTCTCTCTCTCTCTCTCCCTCTCTCTCTCTCTTTGCTAGACGTATGCA
CTCACAAATGTACAATGTTGCCCACCATCTCTCTCTCTCCTTACCTTCCCTTTACccgAC
GTGTGTGTTCTCAGTACGAT
KM 120 (complete)
GAAATCCAGTTGCCCTCATTTCCTCTTCCTCCCCATGGAGACCAGACCCATGGGCGGATG
GATGGATGCATGAATGATGGATAGATGGATGGCGGATGGATGGACGATGGATGAATGGTG
GATGGATGGATAGATGACGGCTGGATGGATGCACGCATGGACGGATGATGGATGGAAGAT
GGATGATGGATGATGGATGGATGATGGATGGATGATGATGCATGTATGGATGGATGATGG
ATGGATGGGTGATGGATGAAGAATTGACGATGGGTGGATGGATGAATTGATGAGAGGATG
GATGGATGGATGGGTTGATGGGTAAGTGGATAGATGGG
KM121 (incomplete)
GTGGCTQGTGGGTTAGCTGACTAGCTAGCTGTCTGCTGTTTGTCTGGCTGCCTGACTCCC
TGTTTGTCTgGCTGGCAGTTTGTCTGGCTGGCTGGCTGGcTGtCTGGCTGGTTGGCTGTC
TGTCTGTCTGTCTGTCTGTCTGTCTGTCTGTCTGGCTGTCTTTCTGTCTGTCTG(SCTAGC
TGGTTGGCTGTCTAGCTGGCTGGTTGGCTGGCTGTGTGGCTGGTTGGCTGTCTG [symbol] CTGTC
TGTCTGTCTGGCTGCCTQGCTGTCTGTCTGTCTGTCTGTCTGTCTGGCTGCCTGGCTGTC
TTTCTGTCTGTCTGGctaGctGGtTGGCTATCTCCCTTCTGCTAGCAAGGCCTTAAATCA
CTAGTGAATTCgcGGccGcCTGCAGGTCGACCATAtggGAGAGCTCCCAACGCGTTggAT
GCatagCTTGAGTATTCTATAGTGtcaCCTAAATagcTTGgCGTAATCATGGTcaTAGCT
GTTT
KM123 (complete)
AAATATATCGATAGATAGACAGATAGATAGATAGATTGaTAGGtaGATTGATTGATAGAT
AGATAGATaGATAGATAGATTGATAgANcGatAGATAGATAGATaGataGATAGATTGGT
AGATTGATTGaTTGATTGATTGAAAGATAGATAG
KM 124 (incomplete)
CCTGGCGTGCTGCGATTCATGGGGTCGCAAAGAATCAGACATGACTGAGCGAAAGAACTG
AACTGAACTGAACTGAGTGGTTGGATGGCTGAATGGATGGATGGGTAGTTGGGTGGATAG
GTGAGTGGGTGAGTGGATGGATAGAGAGATGGATGGCTGATTTACTAATTCTGGTTGCTA
TAGCCTCCACTTCTAGAAGCAGAAATATGAACAGAAATCCTGTTTTCTGAATACTTTTAG
ACATATAAGAAGCAGGAATCTGTAAACCAGGATGTTCCTATGAGAGTCCTAGGCTGTTTT
GCACATCCAAAGAGGTTTTGATACTTCAGAGAAGGCTCCAAACTTCGGATGCCAATGTAA
AGGAAACCCACCGAGGTTCACTTATAGCTTGTTCACACAGATGTAAAGCCAGCTTTGATT
TTCCCTAAAATCCTGCATGTTTTGCCACTGCTTCGAGGATTTTAGGAGAAGCTACCCTAA
AGACTATGACATTTTTCCCCCTTTGTTTCTAATCATACTAGGAAGCACTGATTTACTTTC
GTAGAGACTTGGCGATGCTTCAAGTTTGCCCACCCCCATGG-VICTACAAAGTGCAGATGG
cAGAGCAgGAGTAAAAACGAGACAGAaa
KM 125 (complete)
GACACAGACCGTGATCΓTCAGAAGCCTGAA&GGACACACTGGAAATTTGAGCCGGAGGGA
AGGAATGAGCGGACTGTCTTCCCCTCCCCTCCGCAGAATGACCTTAAAAGAGAAAAGGAA
AAAAGAAAGGAAGGAAGGAAGGGGGAGAAAGAAACAGAAGAAAGAAAGACAGAGGAGGAG
GGCGCAAGAGAAAGAGAAAGGCAGGAAAGAAGGGCGGGIYIGGAGGAAGGJLA.GGAAGGAAG
AAAAGGAGAGATACAAAGAAATCAGTTCCTCTTGG
KM 126 (complete)
TTATGTTGCGTCAGAGAAGCATTAGATGGCTAGCTAATGGTTGGATGGATGGATGGCTAG
ATGGATGGATGACTAGATAGATGGATGGATGACTAGATGGATGGaTGaccAGATGGATGG
ATGGCTAGATGGATGAATGGCTAGATGGATGAATGGCTAGATGGATGGCTGGCTAGATGG
ATGGATGGCGAGGTGGATGGATGGATAGCTAGATGGACAGATGGATGACTAATGTTTGGT
TGGCTAGGTGGATGGAGTGAAAAAGATTTTTTGTGATC
KM 127 (complete)
GGAGAGTGcaTCACGGAACAACGCGAAgTCTTGTGACTGTTAATGGTGGGAGGGACAGTG
GAGGGTTGAgACAGACAGACAGAGACACGGAgAGACAGACAGAgacagagAGAGAGAGAC
AGACACAGAGAGACAGAGAGGcaGAGACAGAgAGCCAGACAGAGACAGAGAGACGGAgAC
AGACAGAGACAGACAGAGACAGGGAAAGACACACAGAGAGAGACCCAgAGAGACAGACCG
GGNTCTAGCCCAGCACGTGTCTGCaCCTGcTGTCCCCAGAGGTAGGAGCACAGGGaTcCT
GGcAGTCGTCAGCCCcTCTTCGCACGGGaacctcgcgcGcaCCATCTTCCCTCCTCACGG
GTGG
KM 128 (complete)
GATCCTTCTCATAAGGTGCAgAcAGt/gCCACACGGGACACACTCCCTGGg/cTCTCTCT
TCCTTCCTTCCTTCCTTCCATCCTTCCTTCTTTCCATCCTTCCCCCTTCCCT_{G}CTTCCTC
CTTCCATCCTTCCTTCTTTAATCCTTCCCTCCTTCCTTCCTTCCTTCCTTCCTTCCTTCT
CCTCCCATCCTTTCTTCCTTCCGTTACGCTATCCTCCACGAGTGTTCCTTAGCATCCTCT
GAAGGAGACCATCCTGGATTCTCCAAAAAAAAAGGAGGGTGTTCCTTGGAGTGTGCTCTT
CACATCTTGCTGATGGGATGATCGTGGATGTCATTCTCCAGCTGCCGCCGCTGCTGTTTG
TTCTCATCTGGtGtGGGTACGTCGTGtaGtGtGtCAGATGGGAGTCTGAT
KM 129 (complete)
CCTCTGCTCTTCCAAGCTAACATTTGCTCCAGGGTCACCCATTGGTTGTCAAGACTGGGT
CTTCTCCCTTTCCCAACACAGATAGACAGACAGACAGACAGACACACACACACACACATA
CACACAGACACACAAACACAGACACACACACACTCTCCCCCTCAGGTGGAGACAGGAACT
GGAACTGGAAGAAGGGTTCTGGAATCCCTGCCAGTTGAGATTATGTTGCCTTTCTGTTAG
AGGTGATGTTGAGATCTGGTAGCATTTGGAAAGCAGTAGAGGGTGTTGATGGGCTCCCCA
GGTGGCACTAGGGGTAAAGAACCTGCCTGCtAATGCAGGAGAAGAAGTAAGAGATTTTGG
TTCAATCCCTGGGTTGGGAAGATCCCCTGGAGAAGGCAATGGCACCCCACTCCAGTACTC
TTGCCTGGAAAATCCCATGGATGGAGGAGCCTGGTGGGCTGCAGTCCATGGGGTCGCTGG
GAGTTGGACACGACTCAGTAACTTACTTTCACTTTTCACTTTCATGCATTGGAGAAGGCA
ATGGCAACCCACTCCAGTGTTCTTGCCTGGAGAATCCCAGGGACGGCGGAGCCTGGTGGG
CTGCCATCTATGGGGTCACACAGAGTCGGACACGACTGAAGCGACTTAGCAGCAGCAGCA
GCATCaAGTTTAATATCAACACTTGG
KM 130 (complete)
ATCAGGc/gGGGGAGGGACGGGGCTCCg/aTGAaAGAGAGAGACAGAGACAGACAGACAG
ACaGACAGACAGACAGACAGACAGACAGACAGACAGACAGAGAGTGAGAGAGAGAGAGAG
AAGGTTACAGTACTGGAATGACGCAGAAACCGTCAAAGAGATGATGAAAAGAAGTGCAAT
TGCAGGTJiAACAGAGATGAGGAAG]\hat{ }AGAAGATAAGAAGAGAGAATGAGAAAGAAAGATAC
AAATACAGACAAATACAAA-IATAGATAGATAGACAGATAGATAGATAGATAGATATGATC
KM131 (complete)
GATCAAACATCTCGTACTGGAGGCATTATGGACAATGAA_{G}GAGCGAGGAACAATGAC_{G}TG
CAAAGAAAACTAAAACTTACTGACAGACAGACAAACAGGCAGACAGACAGACAGACAGAC
ATACAAACAGACAGACAGATAGACAGATAGACAGACAGACCGGCATAGTCAAAGGGATTT
CATCTTCTGGACAATAAAGCTTACATAAAA
KW1 132 (complete)
CCTTCGCTTACTGCTTACTGTTTTTGTGCCAATGGCAAGTAAGCAAGCATTAGACAGCAA
GGGTCACCTGTCCTTCCCCAGTAGACCCAGAGCTGGGCACAAGGAAGCTGTTAATTAGTA
TTGTTGGAAAGAAAGAAGGISAGGAACSGAAGGAAGTATGGAGGGAGGGAGAGAGGGAGGAA
GGAAAGGAGGGCA&GGAGAJYKGGGCAAGJUKGGSAGGGAGTAGGGGCAGGGCATGGCTTCC
CTGTGCAGCCAGTTTGGCAAAGTCATGCTGTGTTTTCACATCTCTCATGCACCTTTCTTT
CCTCATTTTTTTTCATCCTACTTTTATCcagtccttcaGCAGCTTACACATTCAGAGCAA
ACGAATT
KM 133 (complete)
GGATGGAGGAGTGGAACAGTGAATGGACAGCAGCCGAGAGAGAGAGGAGCAGCTGGAGAT
GGCGGACGGATGGATGGGCGGGTGGATGGATGGGTGGATGGATGGATGGGCGGATGGATG
AATGGGTGGATGGATTAATGGATGGATGGATGGATTAATGGGTGGATGGATGGATTAATG
GGTGGATGGATGGATGAATGGGTGGATGGATTAATGGATGGATGGGTGGATTAATGGGTG
GATGGATAAATTAATGGGTGGATGGATGGATTAATGGATGGATGGGTGGATTAATGGGTG
GATGGATGGATGAATGGGCGGATGGNNGNNTGGGCGGATGGATGAATGGGCGGATGGATT
AGTGGGTGGATGGATAGACAGTGNGTGAATGTGTGAAAGGATGG
KI4134 (complete)
CCCAGGACACCTTGGAAGAGGAAATGGGAGGAGGGAGCGGTGGGAATAGGTACACCGGGG
GCTCCAGCATTTCCGAAGAAGAGGATAGGAAGTGGGGTAAAGGGGATGGGAAACTTGTCT
AGAAGATGCCTTTGCCCGGCAAACAlCGGATTCAACAAAGACTGTATACTGAGGATGCTGG
TCTTGGAGAAGCAGCTGGAAGGGATAAGGCTGCGGCGGAGGGGGACAGAGTCCATGCCTG
ATTGGACAAATGGATGAATGCGTGGATGGATGGATGGATGGACGGACTAAGTGAGTGA_{G}T
GGTTGCGGGAACCTCAGACGTTCCCAA_{G}TTGGAGCAGCGCGCCCGGCAGGGGT

### Example 2

### Locating Microsatellites in Bovine DNA

Results

A number of repeat elements were located in bovine DNA sequences. The repeat motif is highlighted in blue. From these located sequences, a number of primer sets were developed (highlighted in red, bold, italicised and underlined, and shown at the end of the sequences).

SEQ 2A
AGGGAGAGGAGGCTCCGCTAAGCTCACAAGGAATGAGTGTGTGGAAGGGCCGATGGTCAGGCGTGGGCTT
TGGGAAGTGCCCCCCTCCCCGAAGATTTCAACCCTGGAGGGAAATCGGAGCTCAGTGACTGGCCTTCCTT
GGCCAGGGGAGCAGAGCGCAGGCTGAACACGGACCCTGTGGCATTTGGATCCAACCAGGGACAAGTTCAC
AGTTCCTCAATAAACTCGTGAACAGCACTTAATGTGTGTACGACACAGCTGGATCAGGAGTCGGGTCCAT
CCTAGTGGGGCTTAGAGTCCAGTGACACTAAGTCTCAGCAATAM2I [symbol] εZOεrZEZlεrZS:CTCCTT
CTCAATTGCTGTCTATCTCTCTCTTTTTCTCCTCTCTCCCCTGATCCACCCACCCACCCACCCACCCATC
CATCCACCCACCCACCCACCCACCCACCCATCCATCCACCCATCCACCTACCCATCCATCCACCCACCCA
cccATccATTTTTccATCCATccAcccAcccGTTCACccACccAccrrzairrG-a [symbol] π [symbol] TGC
CCTCTGTGACTCTCCCCGGCCCCCCAAGCCCTCTGAGACCTGCAGCCTGGTCTCGGCCCCCCACCCTCAG
GGACAGCAGCAGGGCAGACAGGTTTCTCTCCCATCTCAGGAGCTGCCATGTCCAGCTGATTGCTGAGGCC
AAATTCAAGGAATTAGCCTGGGTTCTTCTGCGCCTCACACCTCATATTAATCCACTAGAAGTTTCTATCA
CACTTCAGAACTGTTCCAAACGTTCCTAGTTCTCTCCGCCGCTCCTCTGACACCCCAGCCCTCACCACAC
Bos19F: 5′AATCCACTCACCTGTACCTG 3′
Bos1SR: 5′AGAAGACCAGACGGGATAAG 3′
SEQ 2B
GGAATCTGCAGCCTTCTTCCAGGAGTGATGAAGGTGAGGAAACAGGGCCTCAGGAGCCCAGGGAATCCAG
CTTGGGAGAGTTTCCCAGGGTGATTTTCTGGGTTGGTTGGTTTGTTTTGGTTGGAAACGGGAAAAGCTAG
ATCTGTGCAGAACCCACTT/MZKZZZS\hat{ }4ZSIGAZIRCAGAGCTCCGTGTCATGGGAGTAACTGTCT
GCAGACAGGCTTCTCTCCTCAGTGCACCAACACAAGCCCACTGCTTGATATCTCAACACATAGAGGGGTG
GGTGGAGGGGTGGAAGGGTGGGTGGATGGATGGGTGGGTGGATGGATGGATGGATGGATGGGTGGATGAA
TGGATGGGTGGGTGGATGGGTGGGTGGGTGGGTGGGTGGGTAGATGAATGGATGGGTGGGTGGATGGGTG
GGTGGGTGGGTGGGTAGATGAATGGATGGATGAATGGATGGGTGGGTGGATGGGTGGATGGATGGATGGA
TGGGTGAATGGATGGATGGGTGGATGGATGGGTGGGTGGGTGAATGGATGGGTGGGTGGACAGATGGATG
GATGGATGGGTGGATGGATATATGGATGGGTATGGATGCATGGGTAGATGGATGGACCACTGAATATTCT
Ci [symbol] ε [symbol] π [symbol] πizmGTTAATCAGATACATGAGAAAATTATAATGCTTCAAGGTGCCAATATTT
CAACACTCCAAGTAACACAATGATTCAGCCCAAATCCTCAATATTACTTTAAGGAATGACACTCATGAGT
GAGATGTGAGAGTTTTCAGAAGGTTGCAGGCATTGACATTTTTTGGTCCCGAATGACACTGACTCTGCCT
Bos17F: δTTTTCCAAGGCTTGATTCTAS′
Bos17R: 5A GTGAGCGTCAGAGAGAAAG3′
SEQ 2C
CCACACAGATCCCAACTCTZZKZMCrZCZeZIZZCa\hat{ }rCCTGTCCCACTTTGCTCTAAGGAACTTCAA
GAAGCAAAGGCAAAGCATCAGCTCAAGAACATTTGACTATCCATCCATCTGTGCATCCACCTGTCCACCC
ATTCATCCACCCATCCCTACCCATCCATCTACCCATCCACCCACCCACTCATTCCCATTAATCCATCTAT
CCATCCATCCATCCTCATCCATCCATCTGTCCACCCATCCATCCATCCATCCATCCACTCACTCATTCCC
ATTCATACATCTATCCACCCACCCATCCATCTGTCCATCCATCCATCCACCTACCCACCCATCCATCTGT
CCATCCATCCATCCATCCACCCACCCATCCACTCAACGTGTCCATTAACCATCTTCTATGTGTAAGGCAT
TTTGCTTGTTTTGTGAGGACAGATCAAAGGAAATCAAGTTATTGTTTCTATTCAAGAGAGATTTAAACTT
GAAGGGAAGATTGAAGCAGAAGGGGGAACAGGAGAAAGATGGAGATGATATATATAAATATAAGACACAT
AGAAACCCTACCAGGTCATAAATACATCjQOiiCiaWj [symbol] i [symbol] ZrrTCCCCACAAACCACTTCCTTTT
CCAGCCTTCCTCACGTGGCCGTCGTCCCACAGCTGTCTTCACGTAGCCTTTCACTGTATCCATCTCCTGT
CCACCTCTATTGTTGTCAGTTATGCATTTGCCCACTACCTGAGGAGGACTGTACCTTAAACCTGGCATCT
GATGGCAGATCTGGTTCCTAGTCACCTCCTCATCCCTGGAGATGACTCCAGTTTTCAGAGGGAAGGACAC
πCTCAAGGCCTTGGTTTATGCTGAAAACCACTCTTTTAAAAAAAAAAAAAACAACCACTTTTTATTTTG
TATTGGAGTATAGCCGATTAACAAATGTGATAGTTTCAGGTGAACAGTGAAGGGACTCAGTCATACAAGT
ATCCATTCTTCCTCAAACTCCCCTCTGCCACGAGCCAGCGTGAGCCAGCGTGAGGAACTCCGCCCGTGGC
AAAGGTCGTGAGGAAGGAGGCTCGGCATACAAAAAGGCGGGATCGAACCTCAGGAGTCCCCCTGGAAATT
CTCGAGCATCTACCCCCAAAACCAGAGTCTGCCTACTTTACTGCTTTGTGTTCTCACCTACACCTCTGAC
TTTATGGGGGGCGGGGCGCGAGAGACATCAATAACCTCAGATAGGCAGATGACACCACCCTTATGGCAGA
AAGTGAA
BOS3F: 5TTCCAACCTCTGTTTTCCTA3′
BOS3R: 5A GATGATGAGTTTGGTTTGG3′
SEQ 2D
TTCTCTTCTCGTACGTAGGTATTCTGGTCACACACAGAAGTTAAAGATCTAGAGAGAGGCATGTGGTTAG
GAGAATTGGTTATTGCAGAGCGAGGCAGAGCTGAGTTTGCAGTCCAGCTCTGTAGCCTCACCTGTATACT
CTCAGTTAATCCATAGCCTCTCAGTTTTCCCAGCAATAAAAGAGCTAGAATAGTCCTGCTTTCCCCATAG
CATTGTCATAAG\hat{ }4\hat{ }4iεMM2Sffi4εZZAGACAAGTGCTTAGCTTAGGGCTTACATGTTATTATAG
TTGTTATGTCTTTTCTTCCTTCTTCCTTTCCTCTCTCCCTCCCTCCCGACTTCTTTTCTCTCTTTTTTCT
TCCTTCCTGCTCTTTTCTTTCTCTCTTTGTTCCCTTCCTTCTTCCTTTCTTTCCTTTCTTCTTTCTCTTT
CTTTTCTTTCCTTCTTTCCTTCCTTTCATTTCCAACTGCTGCTTTGCCCATCTCGCTAACATCTTCTGAG
42SMεOεMi/fiiZOεTAAGAGGAATATTCAGAATAAAAAGCGTCACTCTCCATTGGCCTTTGAAG
CCCAGGGACAACCATGACGTCACATCTCATCTTCCTCTCCGAATAGAGAAGATTCAAGTGGCCCAATGCT
TTCAGATGGGACGGCAGTGGCGTTAGCATGAGAAACCGGTTAAGGAGAGGTGTGAAGCTCTTCTGTGTTA
GAGACCGTCCCCGATCTGGCCGTCAGCTGCCTTTGGCCTCCTTGTCCTCTGCTTTCTCTCACGAGCTGGC
Bos23F: 5′GAATAAACGAAATGCGAGTCS′
Bos23R: 5′GTGATCTCTTTGTGGTCCATS′

### Example 3

### Location of DNA Microsatellites in Sheep DNA Using Information From Cattle Repeat Regions

Materials/Methods

Primers were designed from cattle genomic sequences which contained a suitable repeat motif. These primers were designed using the software program Primer 3.

As an example, DNA from sheep was PCR amplified using primers BOS3F: 5′ TTCCAACCTCTGTTTTCCTA 3′ and BOS3R: AGATGATGAGTTTGGTTTGG under the following PCR conditions:

| | |
|---|---|
| 95° C. | 5 minutes |
| 35 cycles of 94° C. | 30 seconds |
| 52° C. | 30 seconds |
| 72° C. | 30 seconds |
| one cycle of 72° C. | 10 minutes.. |

PCR was carried out with a final volume of 10 ul, containing: 1 ul of DNA template and 9 ul of PCR master mix containing all four dNTP's, MgCh, forward and reverse primers and PlatinumTaq Polymerase™ (Gibco).

The PCR master mix was made up as 10 ml volumes containing 20 ul of 100 mM dCTP, dGTP, dTTP and dATP (Bmankein), 300 ul of 50 rnM MgCb (Gibco), 100 ul of 20 mg/ml BSA (Gibco) and 8280 ul ultra pure water (Biotech). To 100 ul of master mix, 200 ng of each primer (forward and reverse) and 2 pg of IRD 800 labelled forward primer was added. 5 units of Taq (Invitrogen) was added to each 100 ul of master mix.

The PCR fragments were then subcloned into pGEM Teasy (Promega), transformed into E. coli by electroporation or a similar methodology. The DNA sequence determined on an ABI 3730 DNA sequencer. The DNA sequence obtained was then aligned with the region defined by the PCR primers from >gil67239891)gblAAFC0221 8335.1 1 Bos taurus Con233460, whole genome shotgun sequence.

New primers BOS3.4RF: 5′AAgCAAAATgCCTTACACAT3′ and BOS3.4RR: 5′AgCATCAgCTCAAgAACATT3\ designed to align with conserved DNA regions identified between sheep and cattle, were used for PCR. One primer was labelled with an infrared dye (IRD800) although any fluorescent or radioactive label can be substituted. Sheep and cattle DNA was PCR amplified and analysed on a LiCor DNA fragment analyser.

Results

The sheep DNA region was sequenced, giving the following:

>Sheep clone 4 from Bos 3.
GAGCTCTCCCATATGGTCGACCTGCAGGCGGCCGCGAATTCACTAGTGATTAGATGATGA
GTTTGGTTTGGGATGTTTTTATGACCGGGTAGGGTCTCTATGTGTCTTATATTTATATAT
ATATCATCTCCATCTTTCTCCTGTTCCCCCTTCTCCTTCAATCTTCCCTTCAATTTAAGC
CTCTCTTGAAACAATAACTTGATTTCCTTTGATCTGTCCTAACTAAACAAGCAAJIATGCC
TTACACATAGAAGATGGTTAATGGACATTTGTTGAGTGGATGGGTGGGTGGACGGATGGA
TGGATGAATGGATGGATCGATGGATGGGTGGATGAATGGATGGATGGGTGGATGAATGGA
TGGATGGATGGGTGGGTGGATAGATGTATGAATGGGAATGAGTGAGTGGATGGATGGATG
GATGGATGGATGGATTGGAAGGGGTGAGTGGATGGGTGGATGGATGGATGGGTGGGAGGG
GATGGATGGGTGGATAGGTGGATGGACGGGCAGGGATGGCTGGATAAATGGGTGGACAGT
TACATGCACGGATGGATGCAGAGTCAAATGTTCTTGAGCTGATGCTTTGCCTTTCATTCT
TGAAGTTCCTTAGAACAAAGTGTGACAGGCTAGGAAAACAGAGGTTGGAAAATCGAATTC
CGCGGCGCCATGGCGCGCGCAGCATGCGACGTCGGGCCCAATTCGCCCTATAGTGAGTCG
TATTACAATTCACT

### Example 4

### Identification of Microsatellites in Alpaca by Screening a DNA Library

Whilst this is an example of screening a DNA library, the skilled person would understand that similar techniques could be used to screen BACs, YACs, P1 Bacteriophages, Lambda bacteriophage or cosmid libraries

Materials/Methods

1. Genomic DNA Digestion

20 μg of genomic alpaca DNA was digested to completion with an excess (5 U/μg DNA) of HaeIII enzyme overnight at 37° C. using the following;

| | |
|---|---|
| 10 ul | alpaca DNA (20 ug) |
| 23 μl | water |
| 12 μl | HaeIII (8 U/μl) (Promega, California, USA) |
| 5 μl | buffer C (Promega, California, USA) |

An aliquot was run on a 1% low melting point gel with a 100 bp ladder. The digest was then extracted once with equal volumes of phenol/chloroform. The DNA was precipitated with 2× volume isopropanol overnight at 4° C. and then washed in 200 μl of 70% ethanol. The pellet was dried well and resuspended in 20 μl of distilled water.

2. DNA Size selection

Loading buffer (10 μl) was added to the sample, which was then heated for 10 min at 60° C. The entire sample was loaded while still warm and the digest was run overnight on a large gel tray with broad tooth combs, using a 2% low-melting point agarose gel, with a 100 bp ladder on either side of the DNA. The 100-500 bp fragments were excised from the gel using a sharp sterile scalpel blade and the gel plug was then incubated overnight at −70° C. to disrupt the agarose architecture

The sample was centrifuged at 14000 rpm for 20 min and the supernatant was removed to another tube, DNA was eluted from the supernatant by precipitating overnight at −20° C. in double the volume of isopropanol. The sample was centrifuged again at 14000 rpm for 20 min and washed twice in 70% ethanol to reveal a white pellet of DNA. This pellet was then dried in a 60° C. oven for 5 min and resusupended in 20 μl of TE. A 3 μl aliquot was electrophoresed on a gel with DNA standards and a size ladder to determine the quality and concentration of the digest. The rest was stored at −20° C.

3. Preparation of Digested Plasmid pUC18 Vector

Digestion of 1 μg of pUC18 supercoiled vector (1 μl) with SmaI.

| | |
|---|---|
| Vector (1 μg/μl) | 1 μl |
| 10 × RE digest buffer E | 1 μl |
| Smal enzyme (1 U/μl) | 5 μl |
| sterile water | 3 μl |

The digest was incubated at 37° C. for 30 min, then the restiction enzyme was inactivated by heating the reaction to 65° C. for 15 min.

This plasmid was further treated with Shrimp alkaline phosphatase (Promega) under manufacturer's conditions.

4. Ligation of Plasmid and Insert DNA

The ligation was set up as follows:

| | | |
|---|---|---|
| Vector (Smal digested/Alk Phos pUC18) | 1 | μl (250 ng) |
| Digested DNA Insert | 7 | μl (53 ng) |
| 10 × Ligase buffer (Promega)(with ATP) | 1 | μl |
| T4 DNA ligase (Promega)(2.5 U/μL) | 1 | μi |
| Total Volume | 10 | μl |

The ligation was incubated at 16° C. for 1-4 h. Reactions can be used immediately, or stored at −20° C. until required. The ligated DNA was again precipitated with 4× volume of ice-cold isopropanol at −80° C. for 30 min and then centrifuged at 11000×g for 10 min at 4° C. The supernatant was discarded and the pellet was washed twice with 70% ethanol. After air drying, the pellet was resuspended in 10 μl sterile water and transformed immediately.

5. Bacterial Transformations

Twenty μl of the culture of electrocompetent E. coli (Invitrogen) thawed on ice was transferred to a sterile 1.5 ml microfuge tube. The cuvettes for electroporation were also placed on ice for chilling. Two μl of the ligation reaction was added, mixed and stood on ice for 1 min. The mix was then transferred to the pre-chilled cuvette and electroporated using a pulse of 1.8 kV, 25 μF, and 200 ohms. Successful electroporation was indicated by time constants in the range of 4.2-4.6 msec. Immediately after electroporation, 1 ml of ice-cold SOC media was added to the cuvette, mixed gently, transferred to a sterile 10 ml centrifuge tube and incubated on ice for 1 hour with gentle shaking.

Following incubation, 100 μl of the transformation mix was plated out on LB-Ampicillin (100 μg/ml) plates containing 1 mM IPTG, 1 mM X-gal. After the liquid was absorbed the plate was inverted and incubated at 37° C. overnight.

6. Screening the Plasmid Library

Hybond N+ nylon membranes were carefully laid over the plates and marked with a needle in three positions to preserve orientation. After 1 min, membranes were gently lifted from the plate using forceps, placed colony side up on filter paper and dried for approximately 10 min at 60° C. The plates were incubated at 4° C. until required. The dried membranes were placed in 20% SDS for 10 min to lyse the cells, then rinsed and soaked in transfer buffer for approximately 20 min. Membranes were removed from the transfer buffer, soaked twice for 10 min each in 1 M Tris-HCl, pH 8.0, before being dried for 1 h and either used immediately or placed between filter papers and stored at room temperature until required.

7. Radiolabellina the (CAAA)5 Oligonucleotide

The oligonucleotide (CAAA)5 (10 ng) was radiolabeled using polynucleotide kinase and gamma32P ATP.

8. Hybridising the Probe, Washing and Autoradiography of Membranes

The membrane was then placed in a glass bottle and prehybridised for 1 h with 20 ml of hybridisation buffer. The membrane was unfurled when it was placed in a rotating hybridisation oven (Hybaid) and the rotisserie was activated. Following prehybridisation, the buffer was removed, 10 ml of fresh hybridisation buffer containing the probe was added, and the bottle incubated over night at 45° C. The annealing temperature of the hybridisation experiment is dependent on the melting temperature of the particular probe used.

The membranes were removed from the bottles and placed in a plastic container in a shaking waterbath. Membranes were washed twice with 2×SSC/1% SDS at 45° C. for 15 min, followed by one wash with 1×SSC/1% SDS at 45° C. and lastly with 1×SSC/0.1% SDS at 45° C. for 10 min. Washes were repeated up to three times until the blank was at background count level.

Following washing, membranes were rinsed in 2×SSC, heat sealed in a plastic bag, and exposed to x-ray film (Hyperfilm-MP, Amersham). Positive colonies were picked with a sterile wire and inoculated into 6 ml of LB broth with 50 μg/ml kanamycin and grown overnight on a shaking incubator at 37° C.

Results

The Alpaca DNA detected using the above method was sequenced to determine the repeat region. The sequence obtained is shown below.

>Alpaca 1.2 microsatellite (CAAA)n repeat motif
ATCTCTGCCTGCAAGCrATGGTGGAAGGGAAAGTGGTGAGAGCCCCTTTTCTCTCTCTCAATTTAGATTAGC
AGGAAAAACTATTTGTGGGGCTTGTTCCTTGGATTAACAACTCTTGGGGATTTTTTTCCTGCCAGAGATGGT
CACTGCTTTTCCTTCTTTCTCTCTCTCCCTTTCTCCCTTTCTCCCTTTCTCCCTTTCTCTCTTTCTCTCTCT
CTTTCTCTCTTTCTTTCTTTCTTTCTTTCTTTCTTTCTTTCTTTCTTTCTTTCTTTCCTTTCTTTTCTTTCT
TTCCTTCTTCCTTTCTTTCTTTCTTCTTTCTCCCTCCCTCCCTCCCTCCCTTCCTCTCTTTCTCTCTTTCTC
TCTTTCπTTTGTCASTGAGGAAGAAGAACCATAGGACAGAAGGGAGGGAATGGGCTCTGCTATTTGAGCCA
GTCTCACAGACTGGTGACTTAATGGCTCTCACAGGACAAATATCTATTG

1. A method for detecting a repeat element in a target ruminant nucleic acid sequence, the method comprising the steps of:
(a) contacting a nucleic acid probe capable of hybridizing with a nucleotide sequence flanking said element; and
(b) detecting the complex formed between the probe and the target nucleic acid, wherein the repeat elements are formed of repeating nucleotide sequences of at least 3 nucleotides.

2. The method of claim 1 wherein the repeat elements are formed of repeating nucleotide sequences of at least 4 nucleotides.

3. The method of claim 1 wherein the repeat elements are formed of repeating nucleotide sequences of at least 5 nucleotides.

4. The method of claim 1 wherein the repeat elements are formed of repeating nucleotide sequences of at least 6 nucleotides.

5. The method of claim 1 wherein the repeat elements are formed of repeating nucleotide sequences selected from any one of Tables 1, 2, 3 or 4.

6. The method of claim 1 wherein the probe is selected from group described in the results section of any one of Examples 1, 2 or 3.

7. The method of claim 1 wherein the probe is selected from the group consisting of the nucleotide sequences that are identified by bold, italics and underlining in the clones described in the results section of any one of Examples 1 or 2.

8. A method for detecting a repeat element in a target ruminant nucleic acid sequence, the method comprising the steps of:
a) contacting a nucleic acid probe capable of hybridizing with a nucleotide sequence flanking said element; and
b) detecting the complex formed between the probe and the target nucleic acid
wherein the target ruminant nucleic acid sequence is selected from the group of DNA sequences in the clones described in the results section of any one of Examples 1, 2, 3 or 4.

9. A method for detecting a plurality of repeat elements in a target ruminant nucleic acid sequence, the method comprising the steps of:
a) contacting a plurality of nucleic acid probes capable of hybridizing with nucleotide sequences flanking said elements; and
b) detecting the complexes formed between the probes and the target nucleic acid.

10. The method of claim 8 wherein the detection of a plurality of repeat elements is carried out simultaneously.

11. A nucleic acid probe selected from the group consisting of the probes as described in the results section of any one of Examples 1, 2 or 3.

12. A nucleic acid probe selected from the group consisting of the nucleotide sequences that are identified by bold, italics and underlining in the clones described in the results section of any one of Examples 1 or 2.

13. A method for detecting a repeat element in a target ruminant nucleic acid sequence, the method comprising the steps of:
a) contacting a nucleic acid probe capable of hybridizing with a nucleotide sequence flanking said element; and
b) detecting the complex formed between the probe and the target nucleic acid using DNA amplification.

14. The method of claim 13 wherein the repeat elements are formed of repeating nucleotide sequences of at least 4 nucleotides.

15. The method of claim 13 wherein the repeat elements are formed of repeating nucleotide sequences of at least 5 nucleotides.

16. The method of claim 13 wherein the repeat elements are formed of repeating nucleotide sequences of at least 6 nucleotides.

17. The method of claim 13 wherein the repeat elements are formed of repeating nucleotide sequences selected from any one of Tables 1, 2, 3 or 4.

18. The method of claim 13 wherein the probe is selected from group described in the results section of any one of Examples 1, 2 or 3.

19. The method of claim 13 wherein the probe is selected from the group consisting of the nucleotide sequences that are identified by bold, italics and underlining in the clones described in the results section of any one of Examples 1 or 2.

20. The method of claim 13 wherein the DNA amplification is carried out using PCR.

21. A method for characterising a repeat element in a target ruminant nucleic acid sequence, the method comprising the steps of.
a) contacting a nucleic acid probe capable of hybridizing with a nucleotide sequence flanking said element;
b) extending the complexes formed between the probe and the target nucleic acid and amplifying the sequence containing the repeat element; and
c) characterising the repeat element using the amplification products.

22. The method of claim 21 wherein the repeat element is characterised according to the number of repeats of at least 3 nucleotides.

23. The method of claim 21 wherein the repeat element is characterised according to the number of repeats of at least 4 nucleotides.

24. The method of claim 21 wherein the repeat element is characterised according to the number of repeats of at least 5 nucleotides.

25. The method of claim 21 wherein the repeat element is characterised according to the number of repeats of at least 6 nucleotides.

26. The method of claim 21 wherein the number of repeats is determined by a method selected from the following: sequencing, hybridisation, electrophoretic separation on the basis of length, and single strand conformational polymorphism analysis (SSCP).

27. The method of claim 26 wherein the hybridization assay is chosen from the list comprising: Southern hybridization, Northern hybridization, dot blot hybridization and solid-phase hybridization.

28. The method of claim 27 wherein the hybridization conditions are sufficiently stringent so that there is a significant difference in hybridization intensity between alleles.

29. The method of claim 28 wherein the hybridization is carried out under high stringency conditions.

30. A method for characterising a repeat element in a target ruminant nucleic acid sequence, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element; and
c) characterising the repeat element using the amplification products by contacting said amplification products with a chip comprising at least one probe selected from the group consisting of the probes described in the results section of any one of Examples 1, 2 or 3.

31. A method for characterising a repeat element in a target ruminant nucleic acid sequence, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element; and
c) characterising the repeat element using the amplification products by contacting said amplification products with a chip comprising at least one probe selected from the group consisting of the nucleotide sequences that are identified by bold, italics and underlining in the clones described in the results section of any one of Examples 1 or 2.

32. A chip comprising at least one probe selected from the group consisting of the probes that are described in the results section of any one of Examples 1, 2 or 3 and complements thereof.

33. A chip comprising at least one probe selected from the group consisting of the nucleotide sequences that are identified by bold, italics and underlining in the clones described in the results section of any one of Examples 1 or 2 and complements thereof.

34. A method of detecting an association between a genotype and a phenotype in a ruminant using a repeat element in a target ruminant nucleic acid, the method comprising the steps of:
a) contacting a nucleic acid probe capable of hybridizing with a nucleotide sequence flanking said element;
b) extending the complexes formed between the probe and the target nucleic acid and amplifying the sequence containing the repeat element;
c) characterising the repeat element using the amplification products;
d) determining the frequency of the repeat element in a trait positive population of ruminants;
e) determining the frequency of the repeat element in a control population of ruminants; and
f) determining whether a statistically significant association exists between said genotype and said phenotype.

35. The method of claim 34 wherein the ruminant control population is a trait negative population, or a random population.

36. The method of claim 34 wherein the method is applied to a pooled biological sample derived from each of said populations

37. The method of claim 34 wherein the method is performed separately on biological samples derived from each individual in said population or a sub sample thereof.

38. A kit for detecting a repeat element in a target ruminant nucleic acid sequence, the kit comprising:
a) a nucleic acid probe capable of hybridizing with a nucleotide sequence flanking said element; and
b) means for detecting the complex formed between the probe and the target nucleic acid.

39. The kit of claim 38 wherein said kit contains a plurality of probes selected from the group consisting of the probes described in the results section of any one of Examples 1, 2 or 3.

40. The kit of claim 38 wherein said kit contains a plurality of probes selected from the group consisting of the nucleotide sequences that are identified by bold, italics and underlining in the clones described in the results section of any one of Examples 1 or 2.

41. The kit of claim 38 wherein the probe is labelled with a detectable molecule.

42. The kit of any one of claim 38 wherein the probe is immobilized on a substrate.

43. The kit of any one of claim 38 further comprising one or more of the reagents necessary to carry out DNA amplification such as a polymerase enzyme.

44. A method for identifying a repeat element in a ruminant nucleic acid sample, the method comprising the steps of:
a) contacting a nucleic acid probe or a plurality of nucleic acid probes, designed to hybridise to repeat elements with at least 3 repeats, with the sample; and
b) detecting the hybrid complex formed between the probe and nucleic acid sample.

45. The method of claim 44 wherein the probe is capable of hybridising to 3 to 10 repeats of a repeat element selected from the repeat elements listed in any one of Tables 1, 2, 3 or 4.


SEQUENCE LISTING (83 sequences)

SEQ ID NO: 1 - DNA, 1065 bp, Ovis sp.
  misc_feature (823)..(823): n is a, c, g, or t
gatcccacgtgctacagagccacgaagcccataggcctcgccgatggaatccgtgctctggatcaaaaccaacccggtcagcctcctcccggccccggccggggggcgggcgccggcggctttggtgactctagataacctcgggccgatcccttcaaggaaactcctggggtgactcctgtccagggaatcatccaaatgggcctgtttctgaaaaaggcccgagtcacagctgtgacagattctgtggatcgtggctggctggctggctggctggctggctggctggctggctggctggctggattcccatgagagtctgaggatggaacacatggacagaaaagcatccgattccctttggtcaagaatcggtctcgccttctgcgcctggtgtctttcctacgtctggatgattccctcccccaccccaccccaccccaccccaccccgcccccgctccgctcccagcttgaaggtgctctcaaggtcccgccggaacgctctcttcctctcttcggagcgcccttctgaaggggaacgttttcttccacgtcatcgccccgagacagcttcagcctggccctcccctccacccccgcctccctctctccctcctgctcctcttcctcctcctctgaactcttgagctctcctcgcaccggcctctcaccccacacggtggcagtgttggcctaggtatgctcaggcgtctcctccccgcatcccagtggactgccactggctctctctcgactgcgtcgtcctgggaccatgtgtttcctggccctttctgcgggtggggggagacccggacgggccnggcgggggtgtgggggagcctgcatgcggggggaagggtgggggcagagaggaggaggaggaggtggncgaggaggaggaggagcaggaggacgaggaggaggaacgacacaactcccgaggtgccagtgtgtgcctgtggcccgggaaacagacgacgcaccgggctggctccgaaaaggggatccccgtcctttgcgacccataccctgtgtccttgctatgtcaacatgtcactcgatc

SEQ ID NO: 2 - DNA, 373 bp, Ovis sp.
  misc_feature (14)..(15): n is a, c, g, or t
gatctttcccgctnnanggggnagcttnaggccaacgtgttcactctcctctttgggtttcctcaagaggctttttagcccctcttcccttgctgccataagggtggtgtcatctgcatatctgaggggatccgtttccggaaagacggatacccccacgtcgcttctttctttcttgctccccgtttctctggccgaattccaagtgattcagcctcttttcctccactcgttttcctacgacacgatcccccatgttgtgcaaaaaagcggttagatcatcgacacttcgaacgcacttgcggccccgggttcctcccggggctacgcctgtctgagcgtcgcttggcgatcgccgactcactgaacggag

SEQ ID NO: 3 - DNA, 563 bp, Ovis sp.
gatcgtgtcgctccttttctgttgtctacgtgtttcacggcgagtgagtgagagagtctttcgatggtttgctaggatgtgtgaatgtcgtgagaccatggtacttgtcagccgtggatgaacagaacggcttcagctttcagggtgatctcaagtgcactttccccacccagcggcgcctgcttgggtttgttgtcttcggactttgtcacggtctctacccaggttgagttgtgtcttctctcggtgggggttccgagtgtgtctcctccttttcctttcttgctcctgggcttgcttgtctgcgtctgctttccaaagtcctgctttgttctccgagcagcgctcgccttggtttcgctttgccggcccctccctccctccctccctctctttcgggggagggggggccgggggagtctgcgatgccgctcgctggtgcccctctctccgccgaccccgccccgagcccccaccgcccgccggcgtctccgtggaatgtccccccagcaccccggaatcgcgtgggggagtgagtctccttcgtggcagcctcctgagga

SEQ ID NO: 4 - DNA, 419 bp, Ovis sp.
gatctcgggaagcacagaaagccagagagttgcatgaacctgaccgtcacgctttcagaagccaagggaaccagaaatgaggttcactcgcgtgtgggtctgtctttccacgggacgaatcctctctttgagcagatgagggttccgggggccccgtggagcagagaggatagagagttccctcaggtcccctgctcctcccatgcacgcgcacgctccccaacggtcctaggaacagcctgccccagaggagcgtgctggccacaacccacctccacggagacggagacggcagtgtccgtccgcgtcagtcatcctcgtccagagtccccgggccgtgggccctcgccttcacgcctggcaccgtccgttctgtaggtgtgtgtcgaacctgcccggagccctgtggcatcgtcccg

SEQ ID NO: 5 - DNA, 700 bp, Ovis sp.
  misc_feature (8)..(8): n is a, c, g, or t
gatcatcntcncgctccntngaangcngtcctcnncaaaaatgacccanagcgctgccggcncctgtcctactagtngcatgataaataanacagtcataagtgcggcgacgatagtcatgccccgcgcccaccggaagganctgactgggttgaaggctctcaagggcntcngtcgangctctcncttatgcgactcctgcattnngaagcanccnnttagtaggttgangcngttgagcaccnncgcnncanggaatggtgcatgcaaggagatggngcccannagtcncncggncacggggcctgccaccatacccncgncgaaacaagcgctcatgagcccgaagtggngagcccgatccaaagagtggacaggacggtcaggtgagtgccatatggaaaggaaaggaagncaacccacnaacaccctcccnacggtggttgngttcantccaagatcagntcctttgactagcgttggtacgacggcnaccacnngggggatggagaaacacaacngttggtttcttttggacgangagcccccctctgtgtgtgtgtgtgtgtgtgtgtgtgtgtgtgtgtgtgtgtgtgagagaacgccagagttttcccganagagagagagagagagagagagacagagagagagagatggggatggggatgggaggaggggtgcgtgggtggggcggatc

SEQ ID NO: 6 - DNA, 847 bp, Ovis sp.
  misc_feature (72)..(72): n is a, c, g, or t
gatcatgtaactcgccttgatcgttgggaaccggagctgaatgaagccataccaaacgacgagcgtgacacnacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgagcgaagtgggcaggcagggggccccccgagcagacaccttccttccaaagaaagggagaacagacagacacccagaagcacaagggagacaacaaatcancggcagggctgggccgggctgggctggggctgctgggggtgggggcgggctcacggaagcaccccggggcgttcatctggacattgatcgtgtcgctccttttctgttgtctacgtgtttcacggcgagtgagtgagagagtctttcgatggtttgctaggatgtgtgaatgtcgtgagaccatggtacttgtcagccgtggatgaacagaacggcttcagctttcagggtgatcttggactgaacacaaccaccgtggggagggtgttcgtgggttggcttcctttcctttccctatggcactcacctgaccgtacctgtccactctttggatcctctagagtcgacctgcaggcatgcaagcttgagtattctatagtgtcanctaagnatcaanctt

SEQ ID NO: 7 - DNA, 341 bp, Ovis sp.
gatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaac

SEQ ID NO: 8 - DNA, 781 bp, Ovis sp.
  misc_feature (662)..(662): n is a, c, g, or t
gatcaatgtgtcctgcaattcacattaattctcgcagctagctgcgttcttcatcgacgcacgagccgagtgatcttcacgataagggcagggaatatggagatggagcagcgaccatcagcccaacacatgaaaatcctttccccaatgtggccctgaaggtctattgagtcttcagagagtgatccgtttctggaaagacggatacccccacgtcgcttctttctttcttgctccccgtttctctggccgaattccaagtgattcagcctcttttcctccactcgttttcctacgacacgatcgattcccaaagagaacgttccttcactcaaaaagtctaggattgctccccttcaagacgactctctttccttttctacattccaacgacatggattcatctattcccaggtgcctaaggatatggaggcctggcggccatcacggactcgaccgtgagaaaagccctgtgctcgcgaagactctccagagactcccagactctctgtgctgtttacggtggagagggagccgacgctcgtgtgcgtcgtggcgggagggtgggtgaccctgtcacgcgagctagtctgtcagcagagaggtttgacccgagacgcccttgtcacacccagggccgggcgtgagccgtcatgactggnccgacacgtgaaacacccttcacccacgtcattcctgaccaacccactagactcatcatttctaggtagacgctggctttggggggagagcttggggaacggggggnttcctgaggctt

SEQ ID NO: 9 - DNA, 711 bp, Ovis sp.
  misc_feature (5)..(5): n is a, c, g, or t
tcggnactctcatggntaatccagccagccagccagncagccagccagccagccagccagccagcnagccncgatcgtgtcgctccttttctgttgtctacgtgttnnacggngagtgagtgagagagtctttcgatggtttgctaggatgtgtgaatgtcgtgagaccatggtacttgtcagccgtggatgaacagaacggcttcagctttcagggtgatcctgaactcccacgccaagggaggccctgtgcgtccctgtgtgctggaggacaccgtgctacccacatcttgatcttggactgaacacaaccaccgtggggagggtgttcgtgggttggcttcctttcctttccctatggnactcacctgaccgtcctgtccactctttggatccttgatctccccctcgccctcgaggccatcggtcggtccttttctttctcctcctcctgctccccgtcctcctactcaccctagtttctctccccgcctccccactccccgcccctccacacacacacacacacacacacacacacacacacacacacacacacacacacacacgcaagtcccgctctctcaaatggatctctcgctgacggccgacgttttcctttcgccttctttccttcctcccgtcctgcttcctttccctttgagtgngtgtgtgngtgtgtgngtgtgtgtntgtgagtgtgtgtgtgtt

SEQ ID NO: 10 - DNA, 303 bp, Ovis sp.
atcccctggagaaggaaatggcaacccactccagtactattgcctggaaaatcccatggacagaggagcctggtaggctacagtctatggggtcgctaagagttggacatgactgagcgacttcacttcacttcacttcacttcataaggtattgaaaatgctgagtgctccattccttttaaaggaatttaaatgttttgttgtctttattcctaatgacaagggaccatgatggaatttagacccactgtccgcccacctatccatccatccaggcagccaccatccacctgtccatgatc

SEQ ID NO: 11 - DNA, 403 bp, Ovis sp.
gatcccattgcagccccagctctcatctcctaagtggctggggcgttttgtttactgttactcagcctctatttcctcacacgtacgtgcagatataatgaacacattccagttgtctggctgtagtgttcagttcagttcagtccagtcgctcagtcatgtccgactctttgcgaccctatgaatcgcagcattccaggcctccctgcccatccatctcatgtccatccagtcagtgatgccatccagccatctcatcctctgtcatctctttctcctcctgcccccaatccttcccagcatcagggtcttttccaatgagtcaactcttcacatgaggtagccaaagtattggagtttcagctttagcatcagtccttccaatgaacacccaggactgatc

SEQ ID NO: 12 - DNA, 644 bp, Ovis sp.
gatctctgatagataagcaaaggttagacctgtcctcagaacttttctgtatgctgtgaatggttcagttcagttcagtcgctcagtcgtgtccgactctttgcgacctcatgaattgcagcatgccaggcctccctgtccatcaccagctcccggagttcactcagactcatgttcattgagttgtagttgtaccttttactaaaagttaattactgtcacacacaaagcgtagtaccacttagtaatcatttattaagtgttgttgttcagtcgctaagttgtgtccgattctttgtgaccctaaggactgcagcacgccaaacttctttgtccttcactatctctcagagtttgctcaaactcatgtccattgagttagtgatgccatccatccatcccatcctctgtcatcccctttctcctcccgccttcaatctttcccagcattagggtctcttccaatgaatcggctaaatctattcaaatatatctttcatttacatggtacgcttcatccgacttggaatgattcagaacctttctaaaaataaacactaggtaaagagtaatttcctcccagatacacatatggggaaacagtaagaattcacaggcaaccctgggagtaaacagaatggatc

SEQ ID NO: 13 - DNA, 251 bp, Ovis sp.
  misc_feature (81)..(81): n is a, c, g, or t
gatcccatggaatcgcagcacgcctggcctccctgttcatcaccatctcccagagttcactcagactcacgtccattgagncagtgatgccatccagccatctcatcctctgtcatccccttctcctcctgcccccaatccctcccagcatcagagtcttttccaatgagtcgactcttcgcatgaggtggccaaagnactggagtttcagcttcagcatcattccttccaaagaaatcccagggctgatc

SEQ ID NO: 14 - DNA, 255 bp, Ovis sp.
gatccctacattgtatttcctagaattttataaaagtagaatcatatagtctgaaaaaaatctttgtatggatatatacttttatttctcttacgaaggcaacttttttatgtctttgtcctctctcccttccttccttccttcctaacttctctctccctctctctttaccatgtcgttctacaattgttctggtactatttgttgaaaaagcaaatcacactttcaattttgtcaaaaatgtttgacactctt

SEQ ID NO: 15 - DNA, 130 bp, Ovis sp.
gatcccgtgaactgcagcagtcctagcttccctgtccttccctagctcctagagtttgctacaactcatgtcagttgagtcagtgatgccatccatccatctcatcctctgtctctcctgtctcctcttg

SEQ ID NO: 16 - DNA, 403 bp, Ovis sp.; identical to SEQ ID NO: 11

SEQ ID NO: 17 - DNA, 208 bp, Ovis sp.
  misc_feature (136)..(138): n is a, c, g, or t
atggatggatggatggatggatggatggatggatggatggatggatggatggatggatggatggatggatggatggatggatggatggatggatggatggatggatggatggatggatggatggatggatggatgnnntncagctaggnangccttccttccttccttccttccttccttccttccttccttcntacttnnnttnntt

SEQ ID NO: 18 - DNA, 427 bp, Ovis sp.
gatcccagggacagacctaaaacactgctttacacacagccttggctttcactgttcagccatctctctctaccaatggacagtgagttgtgggggtgaggaccatgcccatatcatttctacatttccacctcccagcaaggcacccaggaggaccctggaataatctgtcagatggatggaaggatagatggatggatggatggacggatggatggacggatggacggacagatgaatggatggatggacagatggatgggtggacggacggatggatgatggatggacagatggatggatggatggatggatggatggacagataggtggacagatgaatggatggacagacagatggatggatggacagacaatggatagatggatggatggatggatggatggatggacagatgggtagatc

SEQ ID NO: 19 - DNA, 598 bp, Ovis sp.
gatcaattattagaactctattgcatatgtccaaaaaatttaagtagagccatcagtccagttcagtttagttcagttcagtcgctcagtcgtgtctgactctttgcgaccccatgaatcgcagcacgccaggcctccctgtccatcaccaactcccggagttcactcagactcacgttcatcaagtcagtgatgccatccagccatctcatcctctgtcgtccccttctcctcctgccctcaatccctcccagcatcagggtcttttccaatgagtcaacccttcttatgaggtgcccaaagtactggagtttcagctttaacatcattccttccaaagaaatcccagggctgatccaaccagtccattctaaaggagatctgttagtgcagggagcccactgtgttgcctgtatgttctgtgtcttggttcagccgctgtggaccctgagtgagctcttcttttgggacgcagctacagttggattatctgggccacatgcgctcatcaagcttcccagttggctcagtggtaaagaatcccctgcaatgcaggagacacagaagcctcgggttcaattcctgggtcagaaagatc

SEQ ID NO: 20 - DNA, 781 bp, Ovis sp.
gatcatattcagaagaaattattaaaaccataaatttctataagggaagcatgggtttcccttgtggctcagctggtgaaagaatccgcctgcaatgcaggagacctgggttcgatccctgggttgggaagatcccctgaagaaggaaacgacagcccactccattactagtgcctggaaaatcccatggacggaagagcctggttaggctgcagtccatgggatcgtaaagagccagacacgactgcgtgacttcactttcactttcataaggggagcatattagttctaaagcattagttaacaacaccttgctgatctttttgcaaaatttcagaaaataattgtatgtgcgctctctctctctctctctctctctctctctctcacacacacacacacacacacacacacagtttcttttctgagggaccttgagagtaagtgatcttaatgcttccctttgcagacagcacaattcggggtgagggggtgttgtccatggtgctgaagttgtcaggggcagaactagaaataatttcttgactgcagtccatttcttttccgtgtgattatgttgcctcatccagtatattgtgggtcagggtcaatctgttgtctcctttgctctgaaatctctgaaatgctcctagggtgcatcctcacgccaaccagcagctgctttctaaaaggagcatttgaatgcaactctgaatcctgaggaggaaatggttttcactgtggtttgaaatcttttctatactctctccacccacgtata

SEQ ID NO: 21 - DNA, 211 bp, Ovis sp.
  misc_feature (137)..(140): n is a, c, g, or t
atggatggatggatggatggatggatggatggatggatggatggatggatggatggatggatggatggatggatggatggatggatggatggatggatggatggatggatggatggatggatggatggatggatggnnnnctgctannnnnnncttccttccttccttccttcctnnntnnntnanttantnnntnnntnnntncntnnnt

SEQ ID NO: 22 - DNA, 28 bp, Ovis sp.
aggccttccttccttccttccttcctta

SEQ ID NO: 23 - DNA, 622 bp, Ovis sp.
aaggaaggaaggaaggaaggaagggggaggtggagggaggggtctctctggctgtctctctaggagtctattcaagtcaaagtatgatagagctggaagggaacttgattccaatgtggtctaagcctgtgctttcatgtagcatatgaatggatcttctatagttgaggtaaggctcaagagatgcttctcaaaagtcacacagcaagagtgttgatatgtcttcttgattctgggtggagtgttcccttccctacgttaggtttcatttgagacatttcacatttccttccatatgtccatccatccacccatccacccatcattgcatctatggttctatccatccatccgcccatccatcgccatccacccatacacccatccatccatcatccatctatccatcatccatccatccatcatccacccatccacccatcattgcatctatggttctatccatccatccatccatccatccattgccatccacccatacacccatcatccatccatccacccattcatccatccatccatccattcattcattcatctatccatccatccatccatccattcatcaccatccacccatccatccatccatccatccata

SEQ ID NO: 24 - DNA, 413 bp, Ovis sp.
aaggaaggaaggaaggaaggaagggggaggtggagggaggggtctctctggctgtctctctaggagtctattcaagtcaaagtatgatagagctggaagggaacttgattccaatgtggtctaagcctgtgctttcatgtagatatgaatggatcttctatagttgaggtaaggctcagagatgcttctcaaaagtcacacagcaagagtgttgatatgtcttcttgattctggtggagtgttcccttccctacgttaggtttcatttgagacatttcacatttccttccatatgtccatccatccacccatccacccatcattgcatctatggttctatccatccatccacccatccatcgccatccacccatacacccatccatccatcatccatctatccatcatccata

SEQ ID NO: 25 - DNA, 347 bp, Ovis sp.
aggatggatgagtggatggaaggaaggaaagatggatgggtgggtaaaaggatggatggatgggtggacagacggaagaagacaagaatggatgaatgcattcatgcatgcaagggtgtgagaccgtcatgggcgctggtcagggaaggcttcagggactggacttggactgaacttggttgagagagagcccagagtggtgggagtctcaggtgtgctgcggaggatccatgactttgtccacaagaccatgctccccccatccagcatgtggtcttccagagtcactgactcagcttctctcctgctctaggacggaacccaggtgccaaggagctgaccagggg

SEQ ID NO: 26 - DNA, 299 bp, Ovis sp.
atcgatagatagatagatagacagatagaaaatagacgtatagatagatagatagatagatagatagatagataaatagatagatagatagatagatagatagatagatagacagagagacagatagatacaaagacagatagacagatagataggtagacagacagacagataggcagatagatagatagatagacagataggcagatagatagatagatagacagatagatagagagagagagagacagacagacagagagactgacactagctgatggcgcaatgaaaagtgatcc

SEQ ID NO: 27 - DNA, 501 bp, Ovis sp.
gatagttagatagactgggtggatggatggatgtatggacagacagatagactggatggatggatggatggatggatggatggataaatagatagactgggtggatggatggatggatagatagacttgatggatggatggatggacagacagataaactagatggatggatggatgaatggatagatgggtagatagactgggtggatggatggatagacagatagatagactgggtggatggatggatggatggacagacagactggatggatggatggatggatagatgggtagatagactgggtggatggatggatggatggatagttagatagactgggtggatggatgtatggatggacagacagatagactggatggatggatggatggatggacagacagactggatggatggatggatggatggatggatgggtggatgggtagatagactgggtggatggatggatggatggatggatggatggatggatg

SEQ ID NO: 28 - DNA, 248 bp, Ovis sp.
agatagccaaccagctagccagacagacagaaagacagccaggcagccagacagacagacagacagacagacagccaggcagcctgacagacagacagacagacagccaaccagccacacagccagccaaccagccagctagacagccaaccagctagccagacagacagaaagacagccagacagacagacagacagacagacagacagacagacagccaaccagacagacagccagccagccagac

SEQ ID NO: 29 - DNA, 508 bp, Ovis sp.
atggatggatggatggacgggcggatggatgggtggacggatgggcagatggatggatgacagatggatggatggatggatggatggatggatggttggacagacagatggataggcagatagatggttgaatggacagatggatggatgcatggatagatgaatggatggatggacggatggacagatggatggacggatagacggatggatggacagatggatggacaggtggacagatggatggatggtgggtggatggatggatggatggatggacagatggatggacagatggatggatggacagacggatggatgggtggatgggcagatggatggatggatggatgggcaggcaggcacttgggaacccacaggtttccccggaagctacaggcaggaggtggcatgtatgtgaatggtagatgggatctgggtgagagaaaggacagaaggtcacacctctggagacccagtgaaccgaggtgcctgatgggtttctaag

SEQ ID NO: 30 - DNA, 288 bp, Ovis sp.
gattcagacaggcagagagattatatgtaccagaagaaatagacagacagagaacatatgtatatacagagacaaacaggcagagattgttgtagaagaacagacaggcagacagacagacggcaaacgagattgtgagggagggacaaagaaccacagagggattataggcctgaggcgatgaagagtgtgtgtttggtgtgaggtcctcgagcgttgagttccccagcagcactcgaccactgaccatctgccacgccccaacctactaccctcctcctccctctt

SEQ ID NO: 31 - DNA, 454 bp, Ovis sp.
aaggggtcgctcctctttgcagctgccgttcatatgtttgggggagtttggctctagagaagccagggtcacgagtttaggctccatgatgtgggggagcagaccaagaaagtaatttggtgctggtctacagcgcctgggcagagctctgtccatgcctgccttggtcctcaggtgggaatcaggatggttcactgtagctccccatgggtgcagataaaactgcttagagcaccagcgtagagagataggcagaaatgatagaatagattagatatagaggatgggtggatgggttaggtgggtagttgcatgcatgggttgaggggtggcttggtggatggatatgaatggatggatggtagctacgtggatggatgtatagatgggtggataggtgaatgtagatgggtagataatagatggatggatggatgatggatggatgaatggg

SEQ ID NO: 32 - DNA, 288 bp, Ovis sp.
gattcagacagacagagagattatatgtaccagaagaaatagacagacagagaacatatgtatatacagagacaaacagacagagattgttgtagaagaacagacagacagacagacagacggcaaacgagattgtgagggagggacaaagaaccgcagagggattataggcctgaggcgatgaagagtgtgtgtttggtgtgaggtcctcgagcgttgagttccccagcagcactcgaccactgaccatctgccacgccccaacctactaccctcctcctccctctt

SEQ ID NO: 33 - DNA, 400 bp, Ovis sp.
acacacaggataatcttcgtaatgtcttcgtagtatgagttgctttgtgcgagcggtggttacagaactgtttgcctgtgcaagactggtagtggaaggctggagtgaaaattccgaagtggtgcgtctaattctatattagcttctgttttttcattatggggtctctcgtgatgtggaagatagtgaaactaaactacgtttcaggattgtatggaagacacgtctctctctctctctctctctctctctctctctcaatctatcttatctatctatctatctcactctgtctgtctatctatctatctatctatctgtctatctgtctatctatctatctatctatctatctatctatctatctatctatctatctatctttctactgactttcggc

SEQ ID NO: 34 - DNA, 490 bp, Ovis sp.
gatagttagatagactgggtggatggatggatgtatggacagacagatagactggatggatggatggatggatggatggatggataaatagatagactgggtggatggatggatggatagatagacttgatggatggatggatggacagacaggtaaactagatggatggatggatgaatggatagatgggtagatagacagggtggatggatggatagacagatagatagactgggtggatggatggatggatggacagacagactggatggatggatggatggatagatgggtagatagactgggtggatggatggatggatggatagttagatagactgggtggatggatggatggatggacagacagatagactggatggatgaatggatggatggacagacagactggatggatggatggatggatggatggatgggtagatagactgggtggatggatggatggatggatggatggatggatga

SEQ ID NO: 35 - DNA, 309 bp, Ovis sp.
ccaatggatgaatgagtggatgggaggatagacagggagatgatgcactgatagacgcagtaaaaagatgggtgagtaaatggatggatgggcagatggaagaatggatggatgggtggatagaaatatgggcaggtaaagggaggaagggatggggagacggatgaatggataggtggataggaagattgctgagtggatggatggatgggtggatggatgaatggatgatggacggtccagtagcaaggtggatgggcgggtggctagatgtatggatggagaggagtgaatgtcaaaaggaagacc

SEQ ID NO: 36 - DNA, 403 bp, Ovis sp.
ggggatggaggagtggaacagtgaatggacagcagccgagagagaggagcagctggagatggcggacgatggatgggcgggtggatggatgggtggatggatggatgggcggatggatgaatgggcggatggattaatggatggatggatggattaatgggtggatggatggattaatgggtggatgggtggatgaatgggtggatggattaatggatggatgggtgggttaatgggtggatggataaattaatgggtggatggatggattaatggatggatgggtggattaatgggtggatggatggatgaatgggcggatggatgaatgggcggatggatgaatgggcggatggattagtgggtggatggatagacagtgagtgaatgagtgaaaggatgg

SEQ ID NO: 37 - DNA, 326 bp, Ovis sp.
accgttcccagttaagtaattcagctgtatcgtgacttgcagaaggtagagagagagagaaagagagagagagagagagaaagggagaaaagatagatagatagatagatagatagagatagagagagggagaaaaggtagatagagagagagagagagagagagagagagagagagaaagggagaaaaggtagatagatagatagatagatagagagagggagaaaaggtagatagagagagagagagagagagagagagagaggagaatacatggcggaagttgaggcgaagagaggacagcagcgagtgtttatgtttgtgcc

SEQ ID NO: 38 - DNA, 259 bp, Ovis sp.
actctcttagtttctgcgatgaactcactattcttatcttttcaaccgacatggcttagactggggcataccttcgcctgtgccatggaggttacagtggagtagaagacagagaaacagacagagaaacaggcagacagacatacagacaaacagagaaacagatacaagacagacagacagacagagcgacagacgaacagaaaagcagacagacagacagagaaacaaacagatagacagactgacaagcagaagc

SEQ ID NO: 39 - DNA, 267 bp, Ovis sp.
atcaaaccagaatattaatgacgagttctgaatttttggtctgtcgacctcttttccttcttttttacctatttctttcctcagtgaagcgaatataatgtctatctgtttatctgcctatctgtctatctatctatctatctatccgtctgtctgtctgtctaccacgcctaccatacataaggtcccgtgttcgagccctggctgttggagggcttgtgttctaaaaaagcgtgcttttatatgcactgtattcgtgtgtgtatc

SEQ ID NO: 40 - DNA, 323 bp, Ovis sp.
atgaaagcacaggcttagaccacattggaatcaagttcccttccagctctatcatactttgacttgaataaactcctagagagacagccagagagacccctccctccacctcccccctccttccttccttccttccttccttaatcgaattcccgcggccgccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaat

SEQ ID NO: 41 - DNA, 232 bp, Ovis sp.
agagagagagacagacagagagagagagagagagacagacagacagacagacagacggacagacagacagacagacagacagacagacagacagacagacagacagagacagagacagtcagacagagactgacagacagagacagagacagtcagacagagacagagacagtcagacagagactgacagacagacagacagacagacagacagacagacagagagtgagtc

SEQ ID NO: 42 - DNA, 413 bp, Ovis sp.
acatatggatagtaacttatatgatgaccaaatgaagaacaagaaatattacgaagtgaaaagaataataaagcaggcgaaccaagaggctgagcagcgttcataaagtcatgataatcatagactgactaattatgggatatgagggtattgatgccttaaacagagagagagagagagagagagagagagagagagagacagagagagagagagagagagacacagacagacagacagacagacagacagacacacagacagacagacagacagagacagagacagaaagatttataatgaatgcaatgcacaatagagagggagatactaataagtcagagaaaacacgtagcatcctgaggcagacctacagatggagcaagtcggtgttgtgaatataaggagagccc

SEQ ID NO: 43 - DNA, 464 bp, Ovis sp.
gagatgaataggtggatggatggagagatgaatgaatagatggatggatggatggatggatggatgacggatggtgatgggtggatgatgggtggatgacgggtgggtgatgggtggatagatgaataggtgggtggatggagagatgaataggtggatggatggatagatggatgaatgactagatgggtgatggatggatgaatagatggatggatggagagatgaatgaataggtggatggatggatgagggatggataggtgaataggtcgatggatggacagatagatggatggatggatgatgggtggatgatggatgaatagatggatggatggatgatggatggatgaatagatggatggatagagagatgaatgaataggcagatggatggatgatggatgtatagatggatggatgaatgaatagatggatggatggataaatggatggatgcc

SEQ ID NO: 44 - DNA, 324 bp, Ovis sp.
  misc_feature (181)..(182): n is a, c, g, or t
atgatgaagccgacgctgaaggtgatggatggagacgcagatgaatacagagggggagagagagagagagagagagagagagagagagagacagacagacagacagacagacagacagacagacagacagacagagacagagagacagagacagagagagacagacagagagacagatganngaccccttggaagnnagaccttctctcagtgatacnntctcnctaanngngacnactcnctctcgatgtcacttcctacngacggaatctgcttctaaacgnanccnacnttnanntaaaactctctctctacaaacnnnnn

SEQ ID NO: 45 - DNA, 529 bp, Ovis sp.
ggatgatgggatggatgagtggatgatgggatgaatgggtgggtagatgatagaatgaatgggtgggtggatgatgggatggatggatgggtggatgatggatggatgggtggatgatgggatgaatggatgggtggatgatgggatggatggatgggtggatgatgagatggatggatgggtggatgatgggatggatggttgggtgggtgatgggatggatgggtggatgatgggatgaatgggtggatgatggctggatgacaggttgacgatgctggatgggtgggtaggaaggctgctatgccctgagtgtttgtgccccaccgggtctcacgtctggactctgggaccaccgtcacactcacctgggtgtaggtctatctggaaattagcgtcgtgagggtttctggcttctgtcctgcgaggtgactgacccagtagtctagtttgtccccaggagcttctgtgcactgaggcatcctcgccgccccagtaactaagcagcaccccactgtcaggtaagggg

SEQ ID NO: 46 - DNA, 319 bp, Ovis sp.
gatcatagcatcagtggcaaatgagattcttaagaaattgctgtctgtgctcagtctgtctgtctgtctgtctgtctctctgtctgtctgtctctgtctgtctgtctgtctgtctgtctgtctgtctgtctgtctatctgtctgtctgtctgtctgtctgtctgtctgtctgtctgtctctctctctctctctctctctctctctctccctctctctctctctttgctagacgtatgcactcacaaatgtacaatgttgcccaccatctctctctctccttaccttccctttacccgacgtgtgtgttctcagtacgat

SEQ ID NO: 47 - DNA, 338 bp, Ovis sp.
gaaatccagttgccctcatttcctcttcctccccatggagaccagacccatgggcggatggatggatgcatgaatgatggatagatggatggcggatggatggacgatggatgaatggtggatggatggatagatgacggctggatggatgcacgcatggacggatgatggatggaagatggatgatggatgatggatggatgatggatggatgatgatgcatgtatggatggatgatggatggatgggtgatggatgaagaattgacgatgggtggatggatgaattgatgagaggatggatggatggatgggttgatgggtaagtggatagatggg

SEQ ID NO: 48 - DNA, 484 bp, Ovis sp.
gtggctggtgggttagctgactagctagctgtctggtgtttgtctggctggctgactggctgtttgtctggctggcagtttgtctggctggctggctggctgtctggctggttggctgtctgtctgtctgtctgtctgtctgtctgtctgtctggctgtctttctgtctgtctggctagctggttggctgtctagctggctggttggctggctgtgtggctggttggctgtctgtctgtctgtctgtctggctgcctggctgtctgtctgtctgtctgtctgtctggctgcctggctgtctttctgtctgtctggctagctggttggctatctcccttctgctagcaaggccttaaatcactagtgaattcgcggccgcctgcaggtcgaccatatgggagagctcccaacgcgttggatgcatagcttgagtattctatagtgtcacctaaatagcttggcgtaatcatggtcatagctgttt

SEQ ID NO: 49 - DNA, 154 bp, Ovis sp.
  misc_feature (88)..(88): n is a, c, g, or t
aaatatatcgatagatagacagatagatagatagattgataggtagattgattgatagatagatagatagatagatagattgatagancgatagatagatagatagatagatagattggtagattgattgattgattgattgaaagatagatag

SEQ ID NO: 50 - DNA, 628 bp, Ovis sp.
cctggcgtgctgcgattcatggggtcgcaaagaatcagacatgactgagcgaaagaactgaactgaactgaactgagtggttggatggctgaatggatggatgggtagttgggtggataggtgagtgggtgagtggatggatagagagatggatggctgatttactaattctggttgctatagcctccacttctagaagcagaaatatgaacagaaatcctgttttctgaatacttttagacatataagaagcaggaatctgtaaaccaggatgttcctatgagagtcctaggctgttttgcacatccaaagaggttttgatacttcagagaaggctccaaacttcggatgccaatgtaaaggaaacccaccgaggttcacttatagcttgttcacacagatgtaaagccagctttgattttccctaaaatcctgcatgttttgccactgcttcgaggattttaggagaagctaccctaaagactatgacatttttccccctttgtttctaatcatactaggaagcactgatttactttcgtagagacttggcgatgcttcaagtttgcccacccccatggatctacaaagtgcagatggcagagcaggagtaaaaacgagacagaaa

SEQ ID NO: 51 - DNA, 275 bp, Ovis sp.
gacacagaccgtgatcttcagaagcctgaaaggacacactggaaatttgagccggagggaaggaatgagcggactgtcttcccctcccctccgcagaatgaccttaaaagagaaaaggaaaaaagaaaggaaggaaggaagggggagaaagaaacagaagaaagaaagacagaggaggagggcgcaagagaaagagaaaggcaggaaagaagggcgggaaggaggaaggaaggaaggaagaaaaggagagatacaaagaaatcagttcctcttgg

SEQ ID NO: 52 - DNA, 278 bp, Ovis sp.
ttatgttgcgtcagagaagcattagatggctagctaatggttggatggatggatggctagatggatggatgactagatagatggatggatgactagatggatggatgaccagatggatggatggctagatggatgaatggctagatggatgaatggctagatggatggctggctagatggatggatggcgaggtggatggatggatagctagatggacagatggatgactaatgtttggttggctaggtggatggagtgaaaaagattttttgtgatc

SEQ ID NO: 53 - DNA, 364 bp, Ovis sp.
  misc_feature (243)..(243): n is a, c, g, or t
ggagagtgcatcacggaacaacgcgaagtcttgtgactgttaatggtgggagggacagtggagggttgagacagacagacagagacacggagagacagacagagacagagagagagagacagacacagagagacagagaggcagagacagagagccagacagagacagagagacggagacagacagagacagacagagacagggaaagacacacagagagagacccagagagacagaccgggntctagcccagcacgtgtctgcacctgctgtccccagaggtaggagcacagggatcctggcagtcgtcagcccctcttcgcacgggaacctcgcgcgcaccatcttccctcctcacgggtgg

SEQ ID NO: 54 - DNA, 408 bp, Ovis sp.
gatccttctcataaggtgcagacagtgccacacgggacacactccctgggctctctcttccttccttccttccttccatccttccttctttccatccttcccccttccctgcttcctccttccatccttccttctttaatccttccctccttccttccttccttccttccttccttctcctcccatcctttcttccttccgttacgctatcctccacgagtgttccttagcatcctctgaaggagaccatcctggattctccaaaaaaaaaggagggtgttccttggagtgtgctcttcacatcttgctgatgggatgatcgtggatgtcattctccagctgccgccgctgctgtttgttctcatctggtgtgggtacgtcgtgtagtgtgtcagatgggagtctgat

SEQ ID NO: 55 - DNA, 686 bp, Ovis sp.
cctctgctcttccaagctaacatttgctccagggtcacccattggttgtcaagactgggtcttctccctttcccaacacagatagacagacagacagacagacacacacacacacacatacacacagacacacaaacacagacacacacacactctccccctcaggtggagacaggaactggaactggaagaagggttctggaatccctgccagttgagattatgttgcctttctgttagaggtgatgttgagatctggtagcatttggaaagcagtagagggtgttgatgggctccccaggtggcactaggggtaaagaacctgcctgctaatgcaggagaagaagtaagagattttggttcaatccctgggttgggaagatcccctggagaaggcaatggcaccccactccagtactcttgcctggaaaatcccatggatggaggagcctggtgggctgcagtccatggggtcgctgggagttggacacgactcagtaacttactttcacttttcactttcatgcattggagaaggcaatggcaacccactccagtgttcttgcctggagaatcccagggacggcggagcctggtgggctgccatctatggggtcacacagagtcggacacgactgaagcgacttagcagcagcagcagcatcaagtttaatatcaacacttgg

SEQ ID NO: 56 - DNA, 298 bp, Ovis sp.
atcaggcgggggagggacggggctccgatgaaagagagagacagagacagacagacagacagacagacagacagacagacagacagacagacagacagagagtgagagagagagagagaaggttacagtactggaatgacgcagaaaccgtcaaagagatgatgaaaagaagtgcaattgcaggtaaacagagatgaggaagaagaagataagaagagagaatgagaaagaaagatacaaatacagacaaatacaaaaatagatagatagacagatagatagatagatagatatgatc

SEQ ID NO: 57 - DNA, 210 bp, Ovis sp.
gatcaaacatctcgtactggaggcattatggacaatgaaggagcgaggaacaatgacgtgcaaagaaaactaaaacttactgacagacagacaaacaggcagacagacagacagacagacatacaaacagacagacagatagacagatagacagacagaccggcatagtcaaagggatttcatcttctggacaataaagcttacataaaa

SEQ ID NO: 58 - DNA, 367 bp, Ovis sp.
ccttcgcttactgcttactgtttttgtgccaatggcaagtaagcaagcattagacagcaagggtcacctgtccttccccagtagacccagagctgggcacaaggaagctgttaattagtattgttggaaagaaagaaggaaggaaggaaggaagtatggagggagggagagagggaggaaggaaaggagggcaaggagaaagggcaagaaggaagggagtaggggcagggcatggcttccctgtgcagccagtttggcaaagtcatgctgtgttttcacatctctcatgcacctttctttcctcattttttttcatcctacttttatccagtccttcagcagcttacacattcagagcaaacgaatt

SEQ ID NO: 59 - DNA, 404 bp, Ovis sp.
  misc_feature (326)..(327): n is a, c, g, or t
ggatggaggagtggaacagtgaatggacagcagccgagagagagaggagcagctggagatggcggacggatggatgggcgggtggatggatgggtggatggatggatgggcggatggatgaatgggtggatggattaatggatggatggatggattaatgggtggatggatggattaatgggtggatggatggatgaatgggtggatggattaatggatggatgggtggattaatgggtggatggataaattaatgggtggatggatggattaatggatggatgggtggattaatgggtggatggatggatgaatgggcggatggnngnntgggcggatggatgaatgggcggatggattagtgggtggatggatagacagtgngtgaatgtgtgaaaggatgg

SEQ ID NO: 60 - DNA, 353 bp, Ovis sp.
cccaggacaccttggaagaggaaatgggaggagggagcggtgggaataggtacaccgggggctccagcatttccgaagaagaggataggaagtggggtaaaggggatgggaaacttgtctagaagatgcctttgcccggcaaacacggattcaacaaagactgtatactgaggatgctggtcttggagaagcagctggaagggataaggctgcggcggagggggacagagtccatgcctgattggacaaatggatgaatgcgtggatggatggatggatggacggactaagtgagtgagtggttgcgggaacctcagacgttcccaagttggagcagcgcgcccggcaggggt

SEQ ID NO: 61 - DNA, 734 bp, Sheep Clone 4 from Bos 3
gagctctcccatatggtcgacctgcaggcggccgcgaattcactagtgattagatgatgagtttggtttgggatgtttttatgaccgggtagggtctctatgtgtcttatatttatatatatatcatctccatctttctcctgttcccccttctgcttcaatcttcccttcaatttaagcctctcttgaaacaataacttgatttcctttgatctgtcctaactaaacaagcaaaatgccttacacatagaagatggttaatggacatttgttgagtggatgggtgggtggacggatggatggatgaatggatggatcgatggatgggtggatgaatggatggatgggtggatgaatggatggatggatgggtgggtggatagatgtatgaatgggaatgagtgagtggatggatggatggatggatggatggattggaaggggtgagtggatgggtggatggatggatgggtgggaggggatggatgggtggataggtggatggacgggcagggatggctggataaatgggtggacagttacatgcacggatggatgcagagtcaaatgttcttgagctgatgctttgcctttcattcttgaagttccttagaacaaagtgtgacaggctaggaaaacagaggttggaaaatcgaattccgcggcgccatggcgcgcgcagcatgcgacgtcgggcccaattcgccctatagtgagtcgtattacaattcact

SEQ ID NO: 62 - DNA, 840 bp, Bovine sp.
agggagaggaggctccgctaagctcacaaggaatgagtgtgtggaagggccgatggtcaggcgtgggctttgggaagtgcccccctccccgaagatttcaaccctggagggaaatcggagctcagtgactggccttccttggccaggggagcagagcgcaggctgaacacggaccctgtggcatttggatccaaccagggacaagttcacagttcctcaataaactcgtgaacagcacttaatgtgtgtacgacacagctggatcaggagtcgggtccatcctagtggggcttagagtccagtgacactaagtctcagcaataaatccactcacctgtacctgcctccttctcaattgctgtctatctctctctttttctcctctctcccctgatccacccacccacccacccacccatccatccacccacccacccacccacccacccatccatccacccatccacctacccatccatccacccacccacccatccatttttccatccatccacccacccgttcacccacccacccttatcccgtctggtcttctctgccctctgtgactctccccggccccccaagccctctgagacctgcagcctggtctcggccccccaccctcagggacagcagcagggcagacaggtttctctcccatctcaggagctgccatgtccagctgattgctgaggccaaattcaaggaattagcctgggttcttctgcgcctcacacctcatattaatccactagaagtttctatcacacttcagaactgttccaaacgttcctagttctctccgccgctcctctgacaccccagccctcaccacac

SEQ ID NO: 63 - DNA, 840 bp, Bovine sp.
ggaatctgcagccttcttccaggagtgatgaaggtgaggaaacagggcctcaggagcccagggaatccagcttgggagagtttcccagggtgattttctgggttggttggtttgttttggttggaaacgggaaaagctagatctgtgcagaacccacttcagtgtttggaatggtcttttcagagctccgtgtcatgggagtaactgtctgcagacaggcttctctcctcagtgcaccaacacaagcccactgcttgatatctcaacacatagaggggtgggtggaggggtggaagggtgggtggatggatgggtgggtggatggatggatggatggatgggtggatgaatggatgggtgggtggatgggtgggtgggtgggtgggtgggtagatgaatggatgggtgggtggatgggtgggtgggtgggtgggtagatgaatggatggatgaatggatgggtgggtggatgggtggatggatggatggatgggtgaatggatggatgggtggatggatgggtgggtgggtgaatggatgggtgggtggacagatggatggatggatgggtggatggatatatggatgggtatggatgcatgggtagatggatggaccactgaatattctcaggacccagaattgctttaagttaatcagatacatgagaaaattataatgcttcaaggtgccaatatttcaacactccaagtaacacaatgattcagcccaaatcctcaatattactttaaggaatgacactcatgagtgagatgtgagagttttcagaaggttgcaggcattgacattttttggtcccgaatgacactgactctgcct

SEQ ID NO: 64 - DNA, 1267 bp, Bovine sp.
ccacacagatcccaactctttccaacctctgttttcctatcctgtcccactttgctctaaggaacttcaagaagcaaaggcaaagcatcagctcaagaacatttgactatccatccatctgtgcatccacctgtccacccattcatccacccatccctacccatccatctacccatccacccacccactcattcccattaatccatctatccatccatccatcctcatccatccatctgtccacccatccatccatccatccatccactcactcattcccattcatacatctatccacccacccatccatctgtccatccatccatccacctacccacccatccatctgtccatccatccatccatccacccacccatccactcaacgtgtccattaaccatcttctatgtgtaaggcattttgcttgttttgtgaggacagatcaaaggaaatcaagttattgtttctattcaagagagatttaaacttgaagggaagattgaagcagaagggggaacaggagaaagatggagatgatatatataaatataagacacatagaaaccctaccaggtcataaatacatcccaaaccaaactcatcatcttccccacaaaccacttccttttccagccttcctcacgtggccgtcgtcccacagctgtcttcacgtagcctttcactgtatccatctcctgtccacctctattgttgtcagttatgcatttgcccactacctgaggaggactgtaccttaaacctggcatctgatggcagatctggttcctagtcacctcctcatccctggagatgactccagttttcagagggaaggacacttctcaaggccttggtttatgctgaaaaccactcttttaaaaaaaaaaaaaacaaccactttttattttgtattggagtatagccgattaacaaatgtgatagtttcaggtgaacagtgaagggactcagtcatacaagtatccattcttcctcaaactcccctctgccacgagccagcgtgagccagcgtgaggaactccgcccgtggcaaaggtcgtgaggaaggaggctcggcatacaaaaaggcgggatcgaacctcaggagtccccctggaaattctcgagcatctacccccaaaaccagagtctgcctactttactgctttgtgttctcacctacacctctgactttatggggggcggggcgcgagagacatcaataacctcagataggcagatgacaccacccttatggcagaaagtgaa

SEQ ID NO: 65 - DNA, 770 bp, Bovine sp.
ttctcttctcgtacgtaggtattctggtcacacacagaagttaaagatctagagagaggcatgtggttaggagaattggttattgcagagcgaggcagagctgagtttgcagtccagctctgtagcctcacctgtatactctcagttaatccatagcctctcagttttcccagcaataaaagagctagaatagtcctgctttccccatagcattgtcataaggaataaacgaaatgcgagtcagacaagtgcttagcttagggcttacatgttattatagttgttatgtcttttcttccttcttcctttcctctctccctccctcccgacttcttttctctcttttttcttccttcctgctcttttctttctctctttgttcccttccttcttcctttctttcctttcttctttctctttcttttctttccttctttccttcctttcatttccaactgctgctttgcccatctcgctaacatcttctgagatggaccacaaagagatcactaagaggaatattcagaataaaaagcgtcactctccattggcctttgaagcccagggacaaccatgacgtcacatctcatcttcctctccgaatagagaagattcaagtggcccaatgctttcagatgggacggcagtggcgttagcatgagaaaccggttaaggagaggtgtgaagctcttctgtgttagagaccgtccccgatctggccgtcagctgcctttggcctccttgtcctctgctttctctcacgagctggc

SEQ ID NO: 66 - DNA, 481 bp, Alpaca sp.
atctctgcctgcaagctatggtggaagggaaagtggtgagagccccttttctctctctcaatttagattagcaggaaaaactatttgtggggcttgttccttggattaacaactcttggggatttttttcctgccagagatggtcactgcttttccttctttctctctctccctttctccctttctccctttctccctttctctctttctctctctctttctctctttctttctttctttctttctttctttctttctttctttctttctttcctttcttttctttctttccttcttcctttctttctttcttctttctccctccctccctccctcccttcctctctttctctctttctctctttctttttgtcastgaggaagaagaaccataggacagaagggagggaatgggctctgctatttgagccagtctcacagactggtgacttaatggctctcacaggacaaatatctattg

SEQ ID NO: 67 - DNA, 20 bp, Sheep
cgattccctttggtcaagaa

SEQ ID NO: 68 - DNA, 20 bp, Sheep
gacgtggaagaaaacgttcc

SEQ ID NO: 69 - DNA, 20 bp, Sheep
tgctttccaaagtcctgctt

SEQ ID NO: 70 - DNA, 18 bp, Sheep
ggacattccacggagacg

SEQ ID NO: 71 - DNA, 20 bp, Sheep
cgtttctggaaagacggata

SEQ ID NO: 72 - DNA, 20 bp, Sheep
ggaaaacgagtggaggaaaa

SEQ ID NO: 73 - DNA, 20 bp, Sheep
ggtacttgtcagccgtggat

SEQ ID NO: 74 - DNA, 20 bp, Bovine
aatccactcacctgtacctg

SEQ ID NO: 75 - DNA, 20 bp, Bovine
agaagaccagacgggataag

SEQ ID NO: 76 - DNA, 20 bp, Bovine
ttttccaaggcttgattcta

SEQ ID NO: 77 - DNA, 20 bp, Bovine
agtgagcgtcagagagaaag

SEQ ID NO: 78 - DNA, 20 bp, bovine
ttccaacctctgttttccta

SEQ ID NO: 79 - DNA, 20 bp, Bovine
agatgatgagtttggtttgg

SEQ ID NO: 80 - DNA, 20 bp, Bovine
gaataaacgaaatgcgagtc

SEQ ID NO: 81 - DNA, 20 bp, Bovine
gtgatctctttgtggtccat

SEQ ID NO: 82 - DNA, 20 bp, Bovine and Sheep
aagcaaaatgccttacacat

SEQ ID NO: 83 - DNA, 20 bp, bovine and sheep
agcatcagctcaagaacatt